(19) United States
(12) Patent Application Publication
(10) Pub. No.: US 2022/0280590 A1
(43) Pub. Date: Sep. 8, 2022

(54) USE OF INHIBITORS OF YAP AND SOX2 FOR THE TREATMENT OF CANCER

(71) Applicant: Georgetown University, Washington, DC (US)

(72) Inventors: Chunling Yi, Washington, DC (US); Shigekazu Murakami, Bethesda, MD (US)

(73) Assignee: Georgetown University, Washington, DC (US)

(21) Appl. No.: 17/636,735

(22) PCT Filed: Aug. 20, 2020
(86) PCT No.: PCT/US2020/047190

§ 371 Date: Feb. 18, 2022

Related U.S. Application Data
(60) Provisional application No. 62/889,333, filed on Aug. 20, 2019.

(51) Int. Cl. A61K 38/00; A61K 31/138; A61K 31/437; A61P 1/18; A61P 35/00

(52) U.S. Cl. CPC A61K 38/005; A61K 31/138; A61K 31/437; A61P 1/18; A61P 35/00

(57) ABSTRACT

Methods of inducing apoptosis and inhibiting proliferation in YAP-dependent cancer cells, involving contacting the cells with one or more inhibitors of YAP and one or more inhibitors of SOX2. In addition, methods of treating or preventing YAP-dependent cancer in subjects, involving administering to the subject one or more inhibitors of YAP and one or more inhibitors of SOX2.

### CROSS-REFERENCE TO RELATED APPLICATIONS

This application claims benefit of U.S. provisional application No. 62/889,333 filed Aug. 20, 2019, the entirety of which is herein incorporated by reference.

### STATEMENT REGARDING FEDERALLY SPONSORED RESEARCH OR DEVELOPMENT

This invention was made with government support under grant number R01 CA187090 awarded by the National Institutes of Health. The government has certain rights in the invention.

### FIELD OF INVENTION

The present invention generally relates to treatments and other methods involving inhibitors of yes-associated protein 1 (YAP) and SOX2.

### BACKGROUND OF THE INVENTION

Yes-associated protein (YAP) is a transcriptional regulator that is pervasively activated in human malignancies. It is a driver of many key attributes of cancer cells, including cell proliferation (Li et al. 2015; Zhao et al. 2007) and migration (Fu et al., 2014), and studies have shown that it promotes tumor development, progression, and metastasis (Zanconato, Cancer Cell 2016). As an example, YAP is shown to be an essential driver of the initiation of pancreatic ductal adenocarcinoma (PDAC), which is the fourth-leading cause of cancer-related death (Ryan et al., 2014), and increased YAP expression is correlated with decreased survival in human PDAC (Murakami et al., 2017). Such results suggest that YAP may be an effective target for treatments and prophylactics of cancer.

### SUMMARY OF THE INVENTION

The present invention relates to uses associated with the inhibition of YAP and inhibition of SOX2.

An aspect of the invention relates to a method of reducing resistance to the effect of a YAP inhibitor on inducing apoptosis of YAP-dependent cancer cells, the method comprising contacting the YAP-dependent cancer cells with one or more inhibitors of SOX2.

An aspect of the invention relates to a method of reducing resistance to the effect of a YAP inhibitor on inhibiting proliferation of YAP-dependent cancer cells, the method comprising contacting the YAP-dependent cancer cells with one or more inhibitors of SOX2.

An aspect of the invention relates to a method of increasing the efficacy of a YAP inhibitor on inducing apoptosis of YAP-dependent cancer cells, the method comprising contacting the YAP-dependent cancer cells with one or more inhibitors of SOX2.

Another aspect of the invention relates to a method of increasing the efficacy of a YAP inhibitor on inhibiting proliferation of YAP-dependent cancer cells, the method comprising contacting the YAP-dependent cancer cells with one or more inhibitors of SOX2.

In some embodiments, the YAP-dependent cancer cells are selected from pancreatic ductal adenocarcinoma cells, pancreatic cancer cells, liver cancer cells, sarcoma cancer cells, esophageal cancer cells, glioma cancer cells, schwannoma cells, head and neck cancer cells, non-small cell lung cancer cells, gastric cancer cells, kidney cancer cells, colorectal cancer cells, bladder cancer cells, breast cancer cells, ovarian cancer cells, uterine cancer cells, prostate cancer cells, and melanoma cancer cells. For example, the YAP-dependent cancer cells are pancreatic ductal adenocarcinoma cells. Alternatively, the YAP-dependent cancer cells are kidney cancer cells, schwannoma cells, breast cancer cells, or liver cancer cells. In certain embodiments, the YAP-dependent cancer cells have a KRAS mutation.

In some embodiments, the YAP inhibitor comprises an inhibitor of tafazzin (TAZ), an inhibitor of the YAP/TAZ pathway, an inhibitor of the binding of YAP to transcriptional enhancer factor (TEF) domain protein (TEAD), or an inhibitor of TEAD. In some embodiments, the one or more inhibitors of SOX comprises a bromodomain and extraterminal domain (BET) inhibitor.

The one or more inhibitors of SOX2 may contact the YAP-dependent cancer cells in combination with the YAP inhibitor. In some embodiments, the one or more inhibitors of SOX2 contacts the YAP-dependent cancer cells concurrently with the YAP inhibitor. In other embodiments, the one or more inhibitors of SOX2 contacts the YAP-dependent cancer cells shortly before or shortly after the YAP inhibitor.

An aspect of the invention relates to a method of reducing resistance to the effect of a YAP inhibitor on treating YAP-dependent cancer in a subject, the method comprising administering to the subject one or more inhibitors of SOX2.

An aspect of the invention relates to a method of reducing resistance to the effect of a YAP inhibitor on preventing YAP-dependent cancer in a subject, the method comprising administering to the subject one or more inhibitors of SOX2.

An aspect of the method relates to a method of increasing the efficacy of a YAP inhibitor on treating YAP-dependent cancer in a subject, the method comprising contacting the YAP-dependent cancer cells with one or more inhibitors of SOX2.

A further aspect of the invention relates to a method of increasing the efficacy of a YAP inhibitor on preventing YAP-dependent cancer in a subject, the method comprising contacting the YAP-dependent cancer cells with one or more inhibitors of SOX2.

In some embodiments, the YAP-dependent cancer is selected from pancreatic ductal adenocarcinoma, pancreatic cancer, liver cancer, sarcoma, esophageal cancer, glioma, head and neck cancer, non-small cell lung cancer, gastric cancer, kidney cancer, colorectal cancer, bladder cancer, breast cancer, ovarian cancer, uterine cancer, prostate cancer, and melanoma. For example, the YAP-dependent cancer is pancreatic ductal adenocarcinoma. In other embodiments, the YAP-dependent cancer is kidney cancer, breast cancer, or liver cancer. In certain embodiments, the YAP-dependent cancer is associated with a KRAS mutation.

In some embodiments, the YAP inhibitor comprises an inhibitor of TAZ, an inhibitor of the YAP/TAZ pathway, an inhibitor of the binding of YAP to TEAD, or an inhibitor of TEAD. In some embodiments, the one or more inhibitors of SOX comprise one or more BET inhibitors.

In some embodiments, the one or more inhibitors of SOX2 is administered to the subject in combination with the YAP inhibitor. In certain embodiments, the one or more inhibitors of SOX2 is administered to the subject concurrently with the YAP inhibitor. In other embodiments, the one or more inhibitors of SOX2 is administered to the subject shortly before or shortly after the YAP inhibitor.

In addition, an aspect of the invention relates to a method of inducing apoptosis of YAP-dependent cancer cells, the method comprising contacting the YAP-dependent cancer cells with one or more inhibitors of YAP and one or more inhibitors of SOX2.

Also, an aspect of the invention relates to a method of inhibiting growth of YAP-dependent cancer cells, the method comprising contacting the YAP-dependent cancer cells with one or more inhibitors of YAP and one or more inhibitors of SOX2.

In some embodiments, the YAP-dependent cancer cells are selected from pancreatic ductal adenocarcinoma cells, pancreatic cancer cells, liver cancer cells, sarcoma cancer cells, esophageal cancer cells, glioma cancer cells, schwannoma cells, head and neck cancer cells, non-small cell lung cancer cells, gastric cancer cells, kidney cancer cells, colorectal cancer cells, bladder cancer cells, breast cancer cells, ovarian cancer cells, uterine cancer cells, prostate cancer cells, and melanoma cancer cells. For example, the YAP-dependent cancer cells are pancreatic ductal adenocarcinoma cells. Alternatively, the YAP-dependent cancer cells are kidney cancer cells, schwannoma cells, breast cancer cells, or liver cancer cells. In certain embodiments, the YAP-dependent cancer cells have a KRAS mutation.

In some embodiments, the one or more inhibitors of YAP comprises comprise one or more inhibitors of TAZ, one or more inhibitors of the YAP/TAZ pathway, one or more inhibitors of the binding of YAP to TEAD, or one or more inhibitors of TEAD. In some embodiments, the one or more inhibitors of SOX2 comprises one or more BET inhibitors.

In some embodiments, the one or more inhibitors of YAP contact the YAP-dependent cancer cells concurrently with the one or more inhibitors of SOX2. In certain embodiments, the one or more inhibitors of YAP and the one or more inhibitors of SOX2 are in the same composition. In other embodiments, the one or more inhibitors of YAP contact the YAP-dependent cancer cells shortly before or shortly after the one or more inhibitors of SOX2.

Moreover, an aspect of the invention relates to a method of treating YAP-dependent cancer in a subject, the method comprising administering to the subject one or more inhibitors of YAP and one or more inhibitors of SOX2.

An aspect of the invention relates to a method of preventing YAP-dependent cancer in a subject, the method comprising administering to the subject one or more inhibitors of YAP and one or more inhibitors of SOX2.

In some embodiments, the YAP-dependent cancer is selected from pancreatic ductal adenocarcinoma, pancreatic cancer, liver cancer, sarcoma, esophageal cancer, glioma, head and neck cancer, non-small cell lung cancer, gastric cancer, kidney cancer, colorectal cancer, bladder cancer, breast cancer, ovarian cancer, uterine cancer, prostate cancer, and melanoma. For example, the YAP-dependent cancer is pancreatic ductal adenocarcinoma. In other embodiments, the YAP-dependent cancer is kidney cancer, breast cancer, or liver cancer. In certain embodiments, the YAP-dependent cancer is associated with a KRAS mutation.

In some embodiments, the one or more inhibitors of YAP comprises comprise one or more inhibitors of TAZ, one or more inhibitors of the YAP/TAZ pathway, one or more inhibitors of the binding of YAP to TEAD, or one or more inhibitors of TEAD. In some embodiments, the one or more inhibitors of SOX2 comprise one or more BET inhibitors.

In some embodiments, the one or more inhibitors of YAP are administered to the subject concurrently with the one or more inhibitors of SOX2. In certain embodiments, the one or more inhibitors of YAP are administered in the same composition as the one or more inhibitors of SOX2. In other embodiments, the one or more inhibitors of YAP are administered shortly before or shortly after the one or more inhibitors of SOX2.

A further aspect of the invention relates to a kit containing a pharmaceutical composition comprising one or more inhibitors of YAP, a pharmaceutical composition comprising one or more inhibitors of SOX2, and a package insert.

Another aspect of the invention relates to a kit containing a pharmaceutical composition comprising one or more inhibitors of YAP and one or more inhibitors of SOX2, and a package insert.

### BRIEF DESCRIPTION OF THE DRAWING FIGURES

The present disclosure will be further explained with reference to the attached drawing figures.

FIGS. 1A-1H show how YAP ablation induced tumor regression and prolonged survival in mice bearing KRAS mutant pancreatic tumors, as discussed in Example 1. FIG. 1A shows the genetic strategy to sequentially activate KRAS^{G12D} and delete YAP in the pancreas via the Flp-FRT and Tamoxifen (TAM)-induced Cre-loxP recombination systems. FIG. 1B shows the experimental design of the animal studies, in which mice were switched to TAM-containing diet only when the tumors become detectable via MRI(KF: FSF-KRAS^{G12D/+}, R26^{FSF-CreER/Dual}, YAP^{+/+}, Pdx1-Flp; KYYF: FSF-KRAS^{G12D/+}, R26^{FSF-CreER/Dual}, YAP^{flox/flox}, Pdx1-Flp). FIG. 1C shows representative images and tumor area quantification of sequential magnetic resonance imaging (MRI) of the pancreatic regions of KF and KYYF mice pre- or 3 months post-TAM treatment (top and middle panels) and representative photographs of pancreata resected from the same two mice after ˜6 months of TAM treatment (bottom panel) (dotted line marks the pancreas in each MRI image; arrows mark visible nodules on MRI images). FIG. 1D shows Kaplan-Meier survival curve of KF (n=10) and KYYF (n=10) mice from the start of TAM treatment. FIG. 1E shows quantification of histopathological stages of KF and KYYF pancreata after being fed for indicated time periods with a TAM-containing diet (+TAM) or a regular diet (−TAM) starting from the time of detection of visible lesions via MRI (KF+TAM (1-3 months): n=12; KF+TAM (6-9 months): n=6; KYYF+TAM (1-3 months): n=12; KYYF+TAM (6-20 months): n=6; KF&KYYF-TAM (6-9 months): n=5). FIG. 1F shows representative images and quantification of immunofluorescence (IF) staining for Cleaved-Caspase 3 (CC3), pH2AX or Ki67 (Green) in combination with tdTomato (red) and DAPI (blue) in KF and KYYF pancreata after ˜1.5 month of TAM treatment (scale bar=100 μm; n=5). FIG. 1G shows representative images and quantification of IF staining for YAP (green), tdTomato (Tm, red) and DAPI (blue) in KF and KYYF pancreata after ˜1.5 months and >6 months of TAM treatment (scale bar=100 μm; n=5). FIG. 1H shows representative immunohistochemistry (IHC) images of tdTomato and YAP in KYYF pancreata and quantification of percent of tdTomato area before, after ˜1.5 months, and >6 months of TAM treatment (scale bar=50 μm). FIG. 1I shows quantification of percent of Tm-positive area in GFP- and Tm-positive area before, after 1.5 months, and 6 months of TAM treatment. (*P<0.05; **P<0.005; ***P<0.0005; error bars indicate standard deviation)

FIG. 2A-2H shows how YAP functioned as a master transcriptional regulator of multiple metabolic pathways that support nucleotide synthesis, as described in Example 1. FIG. 2A shows an illustration of the experimental design of ex vivo studies, in which primary pancreatic tumor cells were isolated from a tumor-bearing KYYF mouse that was not treated with TAM, and subsequently infected in vitro with Ad-CRE (CRE) to induce YAP deletion or Ad-GFP (GFP) as control. FIG. 2B shows relative cell growth rates in YAP⁺ (GFP) and YAP⁺ (CRE) pancreatic tumor cells at 3- and 5-days post infection (n=3). FIG. 2C shows fold difference in median CellROX fluorescence in YAP⁺ (GFP) and YAP⁺ (CRE) pancreatic tumor cells at 3- and 5-days post infection (n=3). FIG. 2D shows percent of Annexin V positive cells in YAP⁺ (GFP) and YAP⁺ (CRE) pancreatic tumor cells at 3- and 5-days post infection (n=3). FIG. 2E shows representative IHC images of Ki67, pErk, and pS6 in KYYF pancreata and quantification of percent of tdTomato area before, after ˜1.5 months and >6 months of TAM treatment (scale bar=50 μm). FIG. 2F shows IHC images of Ki67, Yap, Sirius Red, Tm, Amy, and CK19 in a matched region containing residual ductal lesions of a KYYF pancreas treated for >6 months with TAM (scale bar=100 μm). FIG. 2G shows Western blot analysis of indicated proteins in KYYF cells at different days post GFP or CRE treatment, in which actin was used as the loading control (shown is representative of at least three independent experiments). FIG. 2H shows Western blot analysis of indicated proteins in KYYF cells at different 5 days post GFP or CRE treatment in 1% FBS or 10% FBS containing medium, in which vinculin (Vinc) was used as the loading control (shown is representative of at least three independent experiments).

FIGS. 3A-3M shows how the YAP/TEAD complex directly transcribed Myc and cooperated with Myc in promoting the expression of metabolic enzymes that maintain growth and survival in KRAS mutant pancreatic tumor cells. FIG. 3A shows a graphic representation of chromatin immunoprecipitation (ChIP)-Seq data showing enrichment peaks of H3K27ac, TEAD1, TEAD3, and TEAD4 along the human MYC gene in HepG2, HCT-116, A549, MCF-7 and ECC-1 cells. FIG. 3B shows ChIP and qRT-PCR analysis in pancreatic tumor cells with TEAD3 antibody or IgG control using primers targeting regions on the mouse Myc promoter that correspond to the TEAD-binding peaks (p1-p3) and a 3′UTR region as negative control (nc) as shown in FIG. 3A (n=3). FIG. 3C shows ChIP and qRT-PCR analysis in YAP null pancreatic tumor cells reconstituted with vector control (YAP⁻) or Flag-YAP (YAP⁺) using primers targeting regions on the mouse Myc promoter that correspond to the TEAD-binding peaks (p1-p3) and a 3′UTR region as negative control (nc) as shown in FIG. 3A (n=3). FIG. 3D shows relative endogenous Myc mRNA levels in KYYF cells at 3- and 5-days after GFP or CRE treatment (n=3). FIG. 3E shows Western blot analysis of indicated proteins in KYYF cells at different days post CRE treatment, in which Vinculin (Vinc) was used as the loading control (shown is representative of at least three independent experiments). FIG. 3F shows representative images of IF staining for tdTomato (red), Myc (green), GFP (magenta), and DAPI (blue) in untreated (−TAM) or TAM-treated (+TAM) orthotopic pancreatic tumors (scale bar represents 50 μm). FIG. 3G shows percent of proliferating cells as determined by 5-ethynyl-2′-deoxyuridine (EdU) incorporation assay (left) or apoptotic cells as determined by terminal deoxynucleotidyl transferase dUTP nick end labeling (TUNEL) assay (right) in KYYF cells stably expressing vector control or human MYC at 5 days post infection with Ad-GFP (−) or CRE (+) (n=3). FIG. 3H shows heatmap of relative mRNA levels of indicated metabolic genes in KYYF cells stably expressing vector control or human MYC at 5 days post GFP or CRE treatment (n=3). FIG. 3I shows ChIP and qRT-PCR analysis in pancreatic tumor cells with rabbit IgG(r), rabbit MYC(r), Mouse IgG(m), or mouse TEAD3(m) antibodies using primers targeting the promoters of indicated genes (n=3). FIG. 3J shows representative images of IF staining for YAP (green), Tm (red), and DAPI (blue) in KYYF pancreata after 15 days of TAM treatment (scale bar=100 μm; n=5). FIG. 3K shows a heatmap showing metabolites significantly changed between TAM-treated (+TAM) and untreated (−TAM) orthotopic pancreatic tumors as measured by liquid chromatography with tandem mass spectrometry (LC-MS/MS) (n=4). FIG. 3L shows Venn diagram and representative enrichment peaks of H3K27ac, MYC, and TEAD4 along the promoters of YAP-regulated metabolic genes illustrating the statuses of TEAD4 or MYC binding based on matched published ChIP-seq datasets from HepG2, HCT-116, A549, and K562 cells. FIG. 3M shows a schematic illustrating the different types of transcription control of various metabolic enzymes by YAP/TEAD and/or Myc and possibly additional factors. (*P<0.05; **P<0.005; ***P<0.0005; ns: not significant; error bars indicate standard deviation).

FIGS. 4A-4P shows how upregulation of SOX2 compensated for YAP loss and restored Myc expression, metabolic homeostasis, and survival in a subset of YAP deficient pancreatic tumor cells, as described in Example 1. FIG. 4A shows a heatmap of relative mRNA levels of indicated genes in YAP⁺ parental (P) KYYF cells or Ad-CRE-treated KYYF cells at day 3 (d3), day 5 (d5), and >2 weeks (long term, LT) post infection (n=3). FIG. 4B shows Western blot analysis of indicated proteins in YAP⁺ parental (P) and two long-term YAP-deleted KYYF lines (YAP⁻ LT #1 and #2), in which Vinc was used as the loading control (shown is representative of at least three independent experiments). FIG. 4C shows Log2 FC in mRNA expression of indicated genes in two long-term YAP-deleted (YAP⁻ LT #1 and #2) relative to YAP⁺ parental KYYF cells (n=3). FIG. 4D shows representative IHC images of SOX2 in KF pancreata after ˜1.5 months of TAM treatment and KYYF pancreata after ˜1.5 or >6 months of TAM treatment (scale bars represent 50 μm). FIG. 4E shows Western blot analysis of SOX2 and Myc proteins in YAP⁻ LT KYYF cells at 3 days post infection with lentivirus carrying vector control or two independent SOX2 shRNAs, in which Vinc was used as the loading control (shown is representative of at least three independent experiments). FIG. 4F shows relative mRNA levels of indicated EMT genes as determined by qRT-PCR analysis in YAP⁻ LT KYYF cells at 3 days post infection with lentivirus carrying vector control or two independent SOX2 shRNAs (n=3). FIG. 4G shows percent of apoptotic cells as determined by TUNEL assay in YAP⁻ LT KYYF cells at 5 days post infection with lentivirus carrying vector control or two independent SOX2 shRNAs (n=3). FIG. 4H shows proliferating cells as determined by EdU assay in YAP⁻ LT KYYF cells at 5 days post infection with lentivirus carrying vector control or two independent SOX2 shRNAs (n=3). FIG. 4I shows representative image (left) and quantification (right) of crystal violet staining of YAP⁻ LT KYYF cells at 5 days post infection with lentivirus carrying vector control or two independent SOX2 shRNAs. FIG. 4J shows relative mRNA levels of indicated genes as determined by qRT-PCR analysis in YAP⁻ LT KYYF cells at 3 days post infection with lentivirus carrying vector control or two independent SOX2 shRNAs (n=3). FIG. 4K shows ChIP and qRT-PCR analysis in YAP⁺ and YAP⁻ murine pancreatic tumor cells with SOX2 antibody using primers targeting an enhancer (En), exon 1 (Ex1), exon 2 (Ex2) and 3-UTR (3utr) regions of the Myc gene, normalized to IgG control (n=3). FIG. 4L shows representative flow cytometry plot of CellROX-stained YAP⁺ parental (P) KYYF cells, KYYF cells at 5 days post GFP or CRE treatment, or two long-term YAP-deleted KYYF lines (YAP⁻ LT #1 and #2). FIG. 4M shows Western blot analysis of TAZ in YAP⁺ parental (P) and two long-term YAP-deleted KYYF lines (YAP⁻ LT #1 and #2), in which actin was used as the loading control (shown is representative of at least three independent experiments). FIG. 4N shows representative IHC images of TAZ in KF and KYYF pancreata after ˜6 months of TAM treatment (scale bar=100 μm). FIG. 4O shows growth curve of YAP⁺ and YAP⁻ mouse pancreatic tumor cells expressing vector control or shTAZ (n=3). FIG. 4P shows representative images of IF staining for SMA (green), tdTomato (red), E-Cad (grey), and DAPI (blue) in KYYF pancreata after ˜1.5 months of TAM treatment (scale bar=100 μm). (*P<0.05; **P<0.005; ***P<0.0005; ns: not significant; error bars indicate standard deviation).

FIGS. 5A-5N shows how metabolic-stress-triggered epigenetic reprogramming drove SOX2 upregulation and lineage shift following YAP ablation in pancreatic tumor cells, as described in Example 1. FIG. 5A shows relative mRNA levels of indicated genes in KYYF cells treated with DMSO or 0.5, 2, 5 μM of 5-Azacytidine (5-Aza) for 3 days (n=3). FIG. 5B shows percent of global DNA methylation in KYYF cells at 3- and 14-days post infection with Ad-GFP or CRE (n=3). FIG. 5C shows relative mRNA levels of indicated genes in KYYF cells at 14 days post infection with Ad-GFP or Ad-CRE in the presence or absence of SAM/SAH supplement (nd: not detectable; n=3). FIG. 5D shows experimental design of examining the effects of nutrient stress on KYYF cells. FIG. 5E shows percent of global DNA methylation in KYYF cells incubated for 2 days in normal or —Glc/Gln/Pyr medium, followed by recovery in normal growth medium for additional 8 days (n=3). FIG. 5F shows relative mRNA levels of indicated genes in KYYF cells incubated for 2 days in normal or −Glc/Gln/Pyr medium, followed by recovery in normal growth medium for additional 12 days (n=3). FIG. 5G shows relative mRNA levels of indicated genes in KYYF cells overexpressing MYC or vector control at 14 days post GFP or CRE treatment (n=3). FIG. 5H shows a schematic illustrating the proposed mechanisms of reactivation of SOX2 and acinar lineage genes following YAP ablation from pancreatic tumor cells based on results from this figure. FIG. 5I shows heatmap of relative mRNA levels of indicated pancreatic lineage markers in KYYF cells at indicated times after CRE treatment (n=3). FIG. 5J shows relative SOX2 mRNA levels in KYYF cells at indicated time points after Ad-CRE infection (n=3). FIG. 5K shows relative mRNA levels of indicated genes YAP⁺ and YAP⁻ KYYF cells at 3 days after infection with lentiviruses carrying vector control or SOX2 shRNA #2 (n=3). FIG. 5L shows percent of DNA methylation within the CpG islands of the indicated gene promoters in WT, KF, and KYYF pancreata (n=3). FIG. 5M shows percent of global DNA methylation in KYYF cells at 3 days after treatment of DMSO or 5 μM of 5-Aza (n=3). FIG. 5N shows growth curve of KYYF cells untreated or treated with SAM (50 μM) and SAH (1 μM) after 4 days of infection with Ad-GFP or Ad-CRE (n=3). (*P<0.05; **P<0.005; ***P<0.0005; ns: not significant; error bars indicate standard deviation).

FIGS. 6A-6D show how BET inhibitors blocked PDAC cells from adapting to YAP loss, as described in Example 2. FIG. 6A shows relative ratios of KPYYF cells pretreated with Ad-GFP (GFP⁺YAP⁺) or Ad-CRE (Tm⁺YAP⁻) and co-cultured over indicated time as determined by fluorescence-activated cell sorting (FACS) (n=3). FIG. 6B shows Log2 fold change (FC) of GFP⁺/Tm⁺ ratios from FIG. 6A treated with epigenetic inhibitors versus to DMSO control. FIG. 6C shows Log2 FC in the ratios of parental and YAP-KD Panc1 cells in co-cultures treated with different epigenetic inhibitors relative to DMSO control. FIG. 6D shows a heatmap representing percent of inhibition (Inh) in established isogenic YAP⁺ and YAP⁻ PDAC cells treated with increasing concentrations of indicated BET inhibitors (Miv: mivebresib (aka ABBV-075); OTX: OTX015).

FIG. 7 shows how BET inhibition blocked the expression of pluripotent transcription factors in primary PDAC cells, as described in Example 2. Western blot assay with indicated antibodies in four different primary PDAC lines expressing variable levels of SOX2/SOX5/TWIST2 after 24 hr treatment with DMSO (−) or Miv (+) is shown.

FIG. 8 shows how YAP/TAZ inhibition sensitized multiple cancer cell lines to BET inhibitor, as described in Example 2. FACS analysis of the relative ratios of control (Ctrl, RFP-) or YAP/TAZ-depleted (shY/T, RFP⁺) cancer cells after co-culturing in the presence of vehicle (Veh) or BE inhibitor mivebresib (Miv) was performed (n=3).

### DETAILED DESCRIPTION

The present invention is based, in part, on the unexpected discovery that, while YAP ablation can induce cell death and growth arrest in cancer cells, a large number of cells will experience an upregulation of SOX2 that compensates for YAP loss, resulting in restoration of metabolic homeostasis and cell survival. However, combining inhibition of YAP with inhibition of SOX2 can more effectively—and surprisingly synergistically—induce apoptosis and reduce cell proliferation and prevents the emergence of clones resistant to YAP loss.

Consequently, the present invention relates to the methods involving inhibition of YAP and inhibition of SOX2 to induce apoptosis and inhibit proliferation of YAP-dependent cancer cells and to treat and prevent YAP-dependent cancer. In addition, the present invention relates to methods involving inhibition of SOX2 to reduce resistance to the effects of, and increase the efficacy of, YAP inhibitors for inducing apoptosis and inhibiting proliferation of YAP-dependent cancer cells and for treatment and prevention of YAP-dependent cancer.

### Definitions

The phraseology or terminology in this disclosure is for the purpose of description and not of limitation, such that the terminology or phraseology of the present specification is to be interpreted by the skilled artisan in light of the teachings and guidance.

As used in this specification and the appended claims, the singular forms “a,” “an,” and “the” include plural referents, unless the context clearly dictates otherwise. The terms “a” (or “an”) as well as the terms “one or more” and “at least one” can be used interchangeably.

Furthermore, “and/or” is to be taken as specific disclosure of each of the two specified features or components with or without the other. Thus, the term “and/or” as used in a phrase such as “A and/or B” is intended to include A and B, A or B, A (alone), and B (alone). Likewise, the term “and/or” as used in a phrase such as “A, B, and/or C” is intended to include A, B, and C; A, B, or C; A or B; A or C; B or C; A and B; A and C; B and C; A (alone); B (alone); and C (alone).

Wherever embodiments are described with the language “comprising,” otherwise analogous embodiments described in terms of “consisting of” and/or “consisting essentially of” are included.

Units, prefixes, and symbols are denoted in their Systeme International de Unites (SI) accepted form. Numeric ranges are inclusive of the numbers defining the range, and any individual value provided herein can serve as an endpoint for a range that includes other individual values provided herein. For example, a set of values such as 1, 2, 3, 8, 9, and 10 is also a disclosure of a range of numbers from 1-10, from 1-8, from 3-9, and so forth. Likewise, a disclosed range is a disclosure of each individual value encompassed by the range. For example, a stated range of 5-10 is also a disclosure of 5, 6, 7, 8, 9, and 10.

An “active agent” is an ingredient that is intended to furnish biological activity. The active agent can be in association with one or more other ingredients. For the present invention, “active agents” refers to one or more inhibitors of YAP and one or more inhibitors of SOX2 collectively; “active agent” refer to the one or more inhibitors of YAP or the one or more inhibitors of SOX2; and “active agent(s)” refers to both the one or more inhibitors of YAP and the one or more inhibitors of SOX2 collectively and individually.

An “effective amount” of a therapy is an amount sufficient to carry out a specifically stated purpose, such as to elicit a desired biological or medicinal response in cells or in a subject. Selection of a particular effective dose can be determined (e.g., via clinical trials, modeling, etc.) by those skilled in the art based upon the consideration of several factors, including the disease or condition to be treated or prevented and its severity, the symptoms involved, the subject's body mass and other relevant physical characteristics, the subject's physiological state, the mode of administration, the route of administration, the target site, the administration of other medications, etc.

The term “pharmaceutical composition” refers to a preparation that is in such form as to permit the biological activity of the active ingredient to be effective and which contains no additional components that are unacceptably toxic to a subject to which the composition would be administered. Such composition can be sterile and can comprise a pharmaceutically acceptable carrier, such as physiological saline. Suitable pharmaceutical compositions can comprise one or more of a buffer (e.g., acetate, phosphate or citrate buffer), a surfactant (e.g., polysorbate), a stabilizing agent (e.g., polyol or amino acid), a preservative (e.g., sodium benzoate), and/or other conventional solubilizing or dispersing agents.

A “subject” refers to any “individual” or “animal” or “patient” or “mammal” for whom diagnosis, prognosis, or therapy is desired. Mammalian subjects include humans, domestic animals, farm animals, sports animals, and laboratory animals including, e.g., humans, non-human primates, canines, felines, porcines, bovines, equines, rodents, including rats and mice, rabbits, etc.

An “antagonist” is a substance that prevents, blocks, inhibits, neutralizes, or reduces a biological activity or effect of another molecule, such as a receptor or ligand.

The terms “induce,”” “cause,” and “stimulate” are used interchangeably and refer to any initiation of an occurrence or activity or any increase in, and in some embodiments a statistically significant increase in, occurrence or activity or extent or volume. For example, “induce” can lead to an increase of about 10%, 20%, 30%, 40%, 50%, 60%, 70%, 80%, 90% or 100% in activity or occurrence.

The terms “inhibit,” “block,” “suppress” and “reduce” are used interchangeably and refer to any decrease, in some embodiments, a statistically significant decrease, in occurrence or activity or extent or volume, including full blocking or complete elimination of the occurrence or activity or extent or volume. For example, “inhibition” can refer to a decrease of about 10%, 20%, 30%, 40%, 50%, 60%, 70%, 80%, 90% or 100% in activity or occurrence. As another example, “reduction” can refer to a decrease of about 10%, 20%, 30%, 40%, 50%, 60%, 70%, 80%, 90% or 100% in extent or volume.

Terms such as “treating” or “treatment” or “to treat” or “alleviating” or “to alleviate” refer to therapeutic measures that cure, slow down, lessen symptoms of, and/or halt progression of a diagnosed pathologic condition or disorder. In certain embodiments, a subject is successfully “treated” for a disease or disorder if the subject shows total, partial, or transient alleviation or elimination of at least one symptom or measurable physical parameter associated with the disease or disorder.

### Inhibitors of YAP

YAP, also known as YAP1 or YAP65 , is a transcriptional regulator that activates the transcription of genes involved in cell proliferation and that suppresses apoptotic genes. YAP, along with its paralog, TAZ, are involved in the transduction of signals in the Hippo tumor suppressor pathway. When the pathway is activated, YAP and TAZ are phosphorylated on a serine residue and sequestered in the cytoplasm. When the Hippo pathway is not activated, YAP/TAZ enter the nucleus and regulate gene expression.

For the present invention, inhibitors of YAP may comprise an antagonist of YAP. In some embodiments, the antagonist includes an antagonist of a molecule downstream of YAP. Suitable antagonists include an antibody or fragment thereof, a binding protein, a polypeptide, and any combination thereof. In some embodiments, the antagonist comprises a nucleic acid molecule. Suitable nucleic acid molecules include double stranded ribonucleic acid (dsRNA), small hairpin RNA or short hairpin RNA (shRNA), small interfering RNA (siRNA), or antisense RNA, or any portion thereof. In some embodiments, the antagonist comprises an optimized monoclonal antibody of the target protein.

In some embodiments, the YAP inhibitor targets TAZ or inhibits the YAP/TAZ pathway.

In addition, YAP lacks a DNA-binding domain, and consequently requires DNA-binding partners such as a TEAD, particularly TEAD1-4 (Zanconato et al. 2015). Thus, in some embodiments, an inhibitor of YAP for use in the present invention may be an agent or compound that blocks the binding between YAP and a binding partner such as TEAD, or that inhibits TEAD.

In certain embodiments, the inhibitor of the YAP may be selected from verteporfin, (R)-PFI 2 hydrochloride, CA3 (CAS Registry Number 300802-28-2; 2,7-bis(piperidinosulfonyl)-9H-fluoren-9-one oxime; also known as CIL56), YAP/YAZ inhibitor 1 (as described in WO 2017/058716, which is incorporated by reference), Super-TDU (1-31) (TFA), YAP-TEAD-IN-1 RFA, TED-347, YAP-TEAD-IN-1, Super-TDU 1-31, Super-TDU TFA, (R)-PFI 2 hydrochloride, XMU MP 1, dasatinib, statins, pazopanib, β-adrenergic receptor agonists, dobutamine, latrunculin B, cytochalasin D, actin inhibitors, drugs that act on the cytoskeleton, blebbistatin, botulinum toxin C3, RHO kinase-targeting drugs (e.g., Y27632), tyrosine-protein phosphatase non-receptor type 14, and a combination thereof. Additional YAP inhibitors for use with the present invention include those described in WO 2017/058716 and WO 2019/040380, which are incorporated herein by reference.

Examples of statins for use in the present invention include, but are not limited to, atorvastatin, fluvastatin, lovastatin, pravastatin, rosuvastatin, simvastatin, and pitavastatin.

### Inhibitors of SOX2

SOX2 is a transcription factor that plays a critical role in the maintenance of embryonic and neural stem cells. It is highly expressed throughout development at various stages (Feng et al. 2015) and for various organ groups, including the brain (Zhao et al. 2004), gastrointestinal tract (Que et al. 2007), skin (Driskell et al. 2009), and eye (Taranova et al. 2006).

The SOX2 gene encodes a protein of 317 amino acids having three main domains: high mobility group domain at the N-terminus, dimerization domain at the center, and transactivation (TAD) domain at the C-terminus (Collignon et al. 1996). As a transcription factor, SOX2 recognizes and binds to the promoter of various target genes via its TAD domain to alter their expression (Nowling 2000).

For the present invention, inhibitors of SOX2 may comprise an antagonist of SOX2. In some embodiments, the antagonist includes an antagonist of a molecule downstream of SOX2. Suitable antagonists include an antibody or fragment thereof, a binding protein, a polypeptide, and any combination thereof. In some embodiments, the antagonist comprises a nucleic acid molecule. Suitable nucleic acid molecules include double stranded ribonucleic acid (dsRNA), small hairpin RNA or short hairpin RNA (shRNA), small interfering RNA (siRNA), or antisense RNA, or any portion thereof. In some embodiments, the antagonist comprises an optimized monoclonal antibody of the target protein.

In some embodiments, SOX2 may be inhibited by agents that alter SOX2 gene expression, such as by using a zinc-finger (ZF)-based artificial transcription factor (ATF) may be used to specifically bind to targets that affect SOX2 expression. Examples of such agents include, but are not limited to, ZF-552SKD, ZF-598SKD, and ZF-619SKD, which are ATFs that bind to the proximal SOX2 promoter; and ZF-4203SKD, which is an ATF that binds to the SOX2 enhancer, SRR1 (Stolzenburg et al. 2012).

In some embodiments, SOX2 may be inhibited by a peptide aptamer for SOX2 targeting. Examples of a peptide aptamer include, but are not limited to, P42, which includes a partial fragment of Venus protein, can interact with SOX2 and inhibiting SOX2 downstream genes (Liu et al. 2020).

In some embodiments, SOX2 may be inhibited by agents that target SOX2-DNA binding, which will inhibit SOX transcriptional activity. Examples of an agent that targets SOX2-DNA binding include, but are not limited to, PIP-S2. PIP-S2 is a hairpin pyrrole-imidazole polyamides-based bioactive synthetic DNA-binding inhibitor that competes with SOX2 for its DNA-binding sequence (5′-CTTTGTT-3′) (Taniguchi et al. 2017).

In some embodiments, SOX2 may be inhibited by small molecules targeting signaling pathways that impact SOX2. Examples include, but are not limited to, X-linked inhibitor of apoptosis proteins such as APG-1387 (Ji et al. 2018); inhibitors of histone demethylase LSD1 such as CBB1007 (Zhang et al. 2013); inhibitors of the epidermal growth factor receptor (EGFR)-SRC-protein kinase B (AKT) signaling pathway such as gefitinib, erlotinib, dasatinib, AKT, and inhibitor MK2206; or inhibitors of the fibroblast growth factor (FGFR)-ERK1/2 signaling pathway such as AZD4547 (Singh et al. 2012; Wang et al. 2018).

In some embodiments, SOX2 may be inhibited by agents that target protein degradation to shut down SOX2 expression. Examples of such an agent includes, but is not limited to MLN4924, which is a neddylation inhibitor that blocks SOX2 expression by targeting the FBXW2-MSX2-SOX2 axis (Yin et al. 2019).

In embodiments of the invention, SOX2 may be inhibited by an inhibitor of bromodomain and extraterminal domain (BET) proteins. BET proteins regulate gene transcription and are implicated in the regulation of cell growth, differentiation, and inflammation. The family of BET proteins primarily consist of bromodomain-containing protein 2 (BRD2), bromodomain-containing protein 3 (BRD3), bromodomain-containing protein 4 (BRD4), and bromodomain testis-specific protein 2 (BRDT). Examples of BET inhibitors include, but are not limited to, mivebresib (ABBV-075); I-BET 151 (GSK1210151A), I-BET 762 (GSK525762), OTX-015, TEN-010, CPI-203[28], and CPI-0610, which target both BRD1 and BRD2; olinone, which targets BRD1; RVX-208 and ABBV-744, which targets BRD2; LY294002, which is a dual-kinase-bromodomain inhibitor; and AZD5153, MT-1, and MS645, which are bivalent BET inhibitors.

In some embodiments, the methods or uses of the invention may comprise contacting YAP-dependent cells, or administering to subjects, one or more inhibitors of YAP and one or more inhibitors of BET.

### Pharmaceutical Compositions

The inhibitor of YAP and the inhibitor of SOX may be formulated in pharmaceutical composition comprising the active agent(s) and one or more pharmaceutically acceptable excipients, carriers, diluents, or other additives.

In some embodiments, the compositions may be suitable for parenteral administration. Thus, the composition may comprise, for example, one or more bulking agents (e.g., dextran 40, glycine, lactose, mannitol, trehalose), one or more buffers (e.g., acetate, citrate, histidine, lactate, phosphate, Tris), one or more pH adjusting agents (e.g., hydrochloric acid, acetic acid, nitric acid, potassium hydroxide, sodium hydroxide), and/or one or more diluents (e.g., water, physiological saline). The pH of the composition is preferably between about 3.0 and 9.0. In one embodiment, the pH is between about 3.5 and 8.0, or between about 5.0 and 7.5.

Compositions of the present invention may also be suitable for oral administration, such as in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of the active agent. Such compositions may comprise, for example, fillers or extenders (e.g., starches, lactose, sucrose, glucose, mannitol, silicic acid), binders (e.g., alginates, gelatin, acacia , sucrose, various celluloses, cross-linked polyvinylpyrrolidone, microcrystalline cellulose) disintegrating agents (e.g., agar-agar, calcium carbonate, alginic acid, certain silicates, sodium carbonate, sodium starch glycolate, lightly crosslinked polyvinyl pyrrolidone, corn starch, potato starch, maize starch, croscarmellose sodium, cross-povidone), wetting agents (e.g., cetyl alcohol, glycerol monostearate, poloxamers), and/or lubricants (e.g., talc, calcium stearate, magnesium stearate, solid polyethylene glycols, sodium lauryl sulfate, colloidal silicon dioxide, stearic acid, silica gel).

Compositions of the present invention may alternatively be suitable for other modes of administration, such as transdermal, nasal, rectal, or vaginal.

The pharmaceutical compositions of the present invention may be prepared using methods known in the art. For example, the active agent and the one or more pharmaceutically acceptable excipients, carriers, diluents, etc., may be mixed by simple mixing, or may be mixed with a mixing device continuously, periodically, or a combination thereof. Examples of mixing devices may include, but are not limited to, a magnetic stirrer, shaker, a paddle mixer, homogenizer, and any combination thereof.

### Uses of the Inhibitor of YAP, Homologue TAZ, and/or Functional Partner TEAD in Combination With the Inhibitor of SOX2

An aspect of the present invention relates to inducing apoptosis of YAP-dependent cancer cells using inhibitors of YAP and inhibitors of SOX2. Thus, some embodiments relate to a method of inducing apoptosis of YAP-dependent cancer cells, the method comprising contacting the cancer cells with one or more inhibitors of YAP and one or more inhibitors of SOX2. Some embodiments relate to the use of one or more inhibitors of YAP and one or more inhibitors of SOX2 to induce apoptosis of YAP-dependent cancer cells. Some embodiments relate to one or more inhibitors of YAP and one or more inhibitors of SOX2 for use in inducing apoptosis of YAP-dependent cancer cells. Some embodiments relate to use of one or more inhibitors of YAP and one or more inhibitors of SOX2 in the manufacture of a medicament for inducing apoptosis of YAP-dependent cancer cells.

An aspect of the present invention relates to inhibiting growth of YAP-dependent cancer cells using inhibitors of YAP and inhibitors of SOX2. Thus, some embodiments relate to a method of inhibiting growth of YAP-dependent cancer cells, the method comprising contacting the YAP-dependent cancer cells with one or more inhibitors of YAP and one or more inhibitors of SOX2. Some embodiments relate to the use of one or more inhibitors of YAP and one or more inhibitors of SOX2 to inhibit growth of YAP-dependent cancer cells. Some embodiments relate to one or more inhibitors of YAP and one or more inhibitors of SOX2 for use in inhibiting growth of YAP-dependent cancer cells. Some embodiments relate to use of one or more inhibitors of YAP and one or more inhibitors of SOX2 in the manufacture of a medicament for inhibiting growth of YAP-dependent cancer cells.

An aspect of the present invention relates to reducing resistance to the effects of a YAP inhibitor on YAP-dependent cancer cells, such as the effects of a YAP inhibitor to induce apoptosis in YAP-dependent cancer cells, or the effects of a YAP inhibitor to inhibit proliferation of YAP-dependent cancer cells. Thus, some embodiments relate to a method of reducing resistance to the effects of a YAP inhibitor on YAP-dependent cancer cells, the method comprising contacting the cancer cells with one or more inhibitors of SOX2. Some embodiments relate to the use of one or more inhibitors of SOX2 to reduce resistance to the effects of a YAP inhibitor on YAP-dependent cancer cells. Some embodiments relate to one or more inhibitors of SOX2 for use in reducing resistance to the effects of a YAP inhibitor on YAP-dependent cancer cells. Some embodiments relate to use of one or more inhibitors of SOX2 in the manufacture of a medicament for reducing resistance to the effects of a YAP inhibitor on YAP-dependent cancer cells.

A further aspect of the present invention relates to increasing the efficacy of a YAP inhibitor on YAP-dependent cancer cells, such as the efficacy of a YAP inhibitor to induce apoptosis in YAP-dependent cancer cells, or the efficacy of a YAP inhibitor to inhibit proliferation of YAP-dependent cancer cells. Thus, some embodiments relate to a method of increasing the efficacy of a YAP inhibitor on YAP-dependent cancer cells, the method comprising contacting the cancer cells with one or more inhibitors of SOX2. Some embodiments relate to the use of one or more inhibitors of SOX2 to increase the efficacy of a YAP inhibitor on YAP-dependent cancer cells. Some embodiments relate to one or more inhibitors of SOX2 for use in increasing the efficacy of a YAP inhibitor on YAP-dependent cancer cells. Some embodiments relate to use of one or more inhibitors of SOX2 in the manufacture of a medicament for increasing the efficacy of a YAP inhibitor on YAP-dependent cancer cells.

The methods/uses of inducing apoptosis of YAP-dependent cancer cells or of inhibiting growth of YAP-dependent cancer cells may comprise contacting the YAP-dependent cancer cells with an effective amount of one or more inhibitors of YAP and an effective amount of one or more inhibitors of SOX2. The YAP-dependent cancer cells may be grown in culture, may be extracted from a subject who has these cells, or may be present in a subject.

The methods/uses of reducing the resistance to the effects of a YAP inhibitor or increasing the efficacy of a YAP inhibitor may comprise contacting the YAP-dependent cancer cells with an effective amount of one or more inhibitors of SOX2. The YAP-dependent cancer cells may be grown in culture, may be extracted from a subject who has these cells, or may be present in a subject. The contacting of the cancer cells with one or more inhibitors of SOX2 may be in combination with contacting the cells with the YAP inhibitor.

In embodiments of the invention, the contacting of the cancer cells may be by direct administration, such as by injection of the active agent(s) onto the cells or, in the case where the cells are present in a subject, injection of the active agent(s) to the site (for example, a tumor) where the cells are located, such as by needle. In some embodiments, the contacting of the cells with the active agent(s) may be achieved by indirect administration; for example, in the case where the cells are in a subject, administration of the active agent(s) parenterally (e.g., intravenous, intramuscular, subcutaneous, etc.), orally, transdermally, or via other routes of administration known in the art, to the subject.

In some embodiments, the subject may be a patient, in particular a human patient, such as a human patient who has been diagnosed with or is suspected of having a YAP-dependent cancer.

In some embodiments, the YAP-dependent cancer cells are selected from pancreatic ductal adenocarcinoma cells, pancreatic cancer cells, liver cancer cells, sarcoma cancer cells, esophageal cancer cells, glioma cancer cells, schwannoma cells, head and neck cancer cells, non-small cell lung cancer cells, gastric cancer cells, kidney cancer cells, colorectal cancer cells, bladder cancer cells, breast cancer cells, ovarian cancer cells, uterine cancer cells, prostate cancer cells, and melanoma cancer cells. In certain embodiment, the YAP-dependent cancer cells comprises a KRAS mutation.

In embodiments of the invention, the one or more inhibitors of YAP used to contact the YAP-dependent cancer cells may comprise one or more inhibitors of TAZ, one or more inhibitors of the YAP/TAZ pathway, one or more inhibitors of the binding of YAP to TEAD, or one or more inhibitors of TEAD.

The efficacy of these methods/uses may be evaluated by one or more known measures. For example, to assess the efficacy of these methods/uses that involve inducing apoptosis of the YAP-dependent cancer cells, the extent of which apoptosis is induced may be measured by observing in the cells morphological changes such as blebbing, condensation of chromatin, irregular chromatin destruction, apoptotic body formation, fragmented nuclei, ruptured plasma membranes, vacuole formation, and/or disrupted organelles, using electron microscopy or other imaging techniques. Apoptosis may also be evaluated using genomic methods such as a DNA ladder assay that can assess the state of the cell chromatin; or a comet assay, which can detect DNA damage; or using proteomic methods that can assay the release of cytochrome c, up- or down-regulation of key inhibitory proteins, and the activation of caspases, such as by Western blotting and other gel-based methods. Additional methods include, but are not limited to, spectroscopic techniques such as flow cytometry, annexin V staining, terminal deoxynucleotidyl transferase (Tdt)-mediated dUTP nick-end labeling (TUNEL assay), caspase detection, and measurement of mitochondrial membrane potential; and imaging techniques such as positron emission tomography (PET) that can detect radiolabeled annexin V concentration.

The efficacy of the methods/uses that involve inhibiting YAP-dependent cancer cell proliferation may be assessed by techniques that include, but are not limited to, nucleoside-analog incorporation assays such as the [³H]thymidine ([³H]TdR) incorporation assay and the 5-bromo-2′-deoxyuridine (BrdU) incorporation assay; cell cycle-associated protein assays using, for example, microscope, cytometry or Western blot analysis, for detecting phase-specific proteins such as topoisomerase II alpha, phosphorylated-histone H3, Ki-67, and proliferating cell nuclear antigen; assays that analyze the presence of cytoplasmic proliferation dyes such as carboxyfluorescein diacetate succinimidyl ester; and indirect techniques such as cell counting, viability, and metabolic activity assays.

In some embodiments, the results of the analyses in cells contacted with the active agent(s) may be compared to results from a control sample, e.g., results from analyzing cells that were not contacted with the active agent(s), cells from a subject who was not administered the active agent(s), cells of the same sample that was evaluated prior to the contact with the active agent(s) (e.g., baseline), cells from the same subject prior to administration of the active agent(s) (e.g., baseline), etc. In embodiments in which the methods/uses are to reduce resistance to the effects of a YAP inhibitor or increase the efficacy of a YAP inhibitor, the results from a control sample may further include results from analyzing cells that were contacted with an inhibitor of YAP only.

Another aspect of the present invention relates to treating YAP-dependent cancer in a subject using inhibitors of YAP and inhibitors of SOX2. Thus, some embodiments relate to a method of treating a YAP-dependent cancer in a subject, the method comprising administering to the subject one or more inhibitors of YAP and one or more inhibitors of SOX2. Some embodiments relate to the use of one or more inhibitors of YAP and one or more inhibitors of SOX2 to treat YAP-dependent cancer in a subject. Some embodiments relate to one or more inhibitors of YAP and one or more inhibitors of SOX2 for use in treating YAP-dependent cancer in a subject. Some embodiments relate to use of one or more inhibitors of YAP and one or more inhibitors of SOX2 in the manufacture of a medicament for treating YAP-dependent cancer in a subject.

An aspect of the present invention relates to preventing YAP-dependent cancer in a subject using inhibitors of YAP and inhibitors of SOX2. Thus, some embodiments relate to a method of preventing a YAP-dependent cancer in a subject, the method comprising administering to the subject one or more inhibitors of YAP and one or more inhibitors of SOX2. Some embodiments relate to the use of one or more inhibitors of YAP and one or more inhibitors of SOX2 to prevent YAP-dependent cancer in a subject. Some embodiments relate to one or more inhibitors of YAP and one or more inhibitors of SOX2 for use in preventing YAP-dependent cancer in a subject. Some embodiments relate to use of one or more inhibitors of YAP and one or more inhibitors of SOX2 in the manufacture of a medicament for preventing YAP-dependent cancer in a subject.

An aspect of the present invention relates to reducing resistance to the effects of a YAP inhibitor on YAP-dependent cancer, such as the effects of a YAP inhibitor to treat YAP-dependent cancer or to prevent YAP-dependent cancer. Thus, some embodiments relate to a method of reducing resistance to the effects of a YAP inhibitor on YAP-dependent cancer in a subject, the method comprising administering to the subject one or more inhibitors of SOX2. Some embodiments relate to the use of one or more inhibitors of SOX2 to reduce resistance to the effects of a YAP inhibitor on YAP-dependent cancer in a subject. Some embodiments relate to one or more inhibitors of SOX2 for use in reducing resistance to the effects of a YAP inhibitor on YAP-dependent cancer in a subject. Some embodiments relate to use of one or more inhibitors of SOX2 in the manufacture of a medicament for reducing resistance to the effects of a YAP inhibitor on YAP-dependent cancer in a subject.

An additional aspect of the present invention relates to increasing efficacy of a YAP inhibitor on YAP-dependent cancer. Thus, some embodiments relate to a method of increasing efficacy of a YAP inhibitor on YAP-dependent cancer in a subject, the method comprising administering to the subject one or more inhibitors of SOX2. Some embodiments relate to the use of one or more inhibitors of SOX2 to increase efficacy of a YAP inhibitor on YAP-dependent cancer in a subject. Some embodiments relate to one or more inhibitors of SOX2 for use in increasing efficacy of a YAP inhibitor on YAP-dependent cancer in a subject. Some embodiments relate to use of one or more inhibitors of SOX2 in the manufacture of a medicament for increasing efficacy of a YAP inhibitor on YAP-dependent cancer.

The methods/uses of treating or preventing YAP-dependent cancer in a subject may comprise administering to the subject an effective amount of one or more inhibitors of YAP and an effective amount of one or more inhibitors of SOX2. The methods/uses of reducing resistance to the effects of a YAP inhibitor or increasing efficacy of a YAP inhibitor may comprise administering to the subject an effective amount of one or more inhibitors of SOX2; in certain embodiments the administration of the one or more inhibitors of SOX2 may be in combination with the treatment by the YAP inhibitor. In embodiments of the invention, administration of these active agent(s) to the subject may be parenterally (e.g., intravenous, intramuscular, subcutaneous, etc.), orally, transdermally, or via other routes of administration known in the art.

In some embodiments, the subject may be a patient, in particular a human patient, such as a human patient who has been diagnosed with or is suspected of having a YAP-dependent cancer.

In some embodiments, the YAP-dependent cancer is selected from pancreatic ductal adenocarcinoma, pancreatic cancer, liver cancer, sarcoma, esophageal cancer, glioma, head and neck cancer, non-small cell lung cancer, gastric cancer, kidney cancer, colorectal cancer, bladder cancer, breast cancer, ovarian cancer, uterine cancer, prostate cancer, and melanoma. In some embodiments, the YAP-dependent cancer is associated with a KRAS mutation.

In embodiments of the invention, the one or more inhibitors of YAP administered to the subject may comprise one or more inhibitors of TAZ, one or more inhibitors of the YAP/TAZ pathway, one or more inhibitors of the binding of YAP to TEAD, or one or more inhibitors of TEAD.

Efficacy of treatment of these methods/uses can be evaluated by one or more known measures. For example, those with YAP-dependent cancer cells subjected to methods/uses of the present invention can experience outcomes including extended survival, longer remission, reduced risk of relapse, and/or improved tumor response as compared with the same outcome(s) in those with the same YAP-dependent cancer cells not subjected to methods/uses of the invention, i.e., control patients. An outcome in a subject treated by a method/use of the invention can be compared, for example, to the median outcome in a population of control patients. The population of control patients can be administered, for example, a regimen selected from the group consisting of a placebo, surgery, radiation, chemotherapy, targeted therapy, and combinations thereof. Comparisons can be analyzed statistically using, for example, the Wilcoxon signed rank test.

In some embodiments, outcome in a subject with YAP-dependent cancer cells receiving active agent(s) according to the invention may be compared with median outcome in subjects with the same YAP-dependent cancer cells receiving a placebo. In some embodiments, outcome in a subject with YAP-dependent cancer cells receiving active agent(s) according to the invention is compared with median outcome in subjects with the same YAP-dependent cancer cells receiving surgery, radiation, chemotherapy, targeted therapy or a combination thereof. In some embodiments, outcome in a subject with YAP-dependent cancer cells receiving active agent(s) according to the invention is compared with median outcome in subjects with the same YAP-dependent cancer cells receiving a standard treatment regimen. In embodiments in which the methods/uses relate to reducing resistance to the effects of a YAP inhibitor or increasing the efficacy of a YAP inhibitor, outcome in a subject receiving one or more inhibitors of SOX2 according to the invention is compared with median outcome in subjects who are treated with the YAP inhibitor without administration of one or more inhibitors of SOX2.

In some embodiments, response to administration of active agent(s) according to the invention compares one or more measures of efficacy after administration of active agent(s) according to the invention, to baseline, e.g., prior to administration of active agent(s) according to the invention. A baseline assessment is preferably performed within 24, 48, or 72 hours, or within 1, 2, 3, or 4 weeks prior to the first administration of active agent(s) according to the invention. In certain embodiments, a baseline assessment is performed within 24 hours prior to the first administration active agent(s) according to the invention.

“Tumor burden” is the total mass or total size of cancerous tissue in a subject's body. Tumor response can be evaluated by measures including objective response rate, disease control rate, and duration of response.

Objective response rate assesses reduction of tumor size, for example, tumor diameter, which can be determined by clinical examination and/or imaging. Where a subject has multiple tumors, tumor size can optionally be expressed as the average diameter of all tumors. Imaging methods include computed tomography (CT); MRI; and PET, such as (18)F-fluorodeoxyglucose PET. In some embodiments, MRI, in particular, gadolinium-enhanced MRI, is utilized to assess tumor response. Accordingly, in one aspect, the invention provides a method of reducing tumor burden, i.e., tumor mass and/or tumor size, in a subject having YAP-dependent cancer, the method comprising administering to the patient one or more inhibitors of YAP and one or more inhibitors of SOX2. Reduction in tumor burden may be measured relative to baseline.

Duration of response is the length of time from the achievement of a response until disease progression, i.e., the period in which a tumor does not grow or spread, or death. Duration of response in patients receiving the active agent(s) can be, for example, at least 4, 6, 8, 10, or 12 weeks, at least 4, 6, 8, 10, 12, 16, 18, or 24 months, or at least 3, 4, or 5 years. Accordingly, in one aspect, the invention provides a method of increasing the duration of response in subject having YAP-dependent cancer, the method comprising administering to the patient one or more inhibitors of YAP and one or more inhibitors of SOX2. Increase in duration of response is measured relative to the median duration of response in a control population.

Survival can be assessed as overall survival, i.e., the length of time a patient lives, or as progression-free survival, i.e., the length of time a patient is treated without progression or worsening of the disease. Survival can be measured from the date of diagnosis or from the date that treatment commences. Overall survival, median overall survival, progression-free survival, and median progression-free survival can be calculated, for example, by Kaplan-Meier analysis, based on the response to treatment. Accordingly, in one aspect, the invention provides a method of increasing overall survival in a subject having YAP-dependent cancer, the method comprising administering to the patient one or more inhibitors of YAP and one or more inhibitors of SOX2. Increase in overall survival is measured relative to the median overall survival in a control population. In another aspect, the invention provides a method of increasing progression-free survival in a subject having YAP-dependent cancer, the method comprising administering to the patient one or more inhibitors of YAP and one or more inhibitors of SOX2. Increase in progression-free survival is measured relative to the median progression-free survival in a control population.

A patient is successfully treated according to the methods of the invention if the patient experiences or displays at least one of the following outcomes after administration of one or more inhibitors of YAP and one or more inhibitors of SOX2:
undetectability of the tumor (or at least one tumor, if multiple tumors are present at baseline);
at least about 10%, 20%, 30%, 40%, 50%, 60%, 70%, 80%, or 90% reduction in tumor size compared to baseline;
no significant increase in tumor size compared to baseline;
significantly increased duration of response compared with median duration of response of a population of control patients;
significantly increased progression-free compared with median progression-free survival of a population of control patients;
significantly increased overall survival compared with median overall survival of a population of control patients.

The one or more inhibitors of YAP and the one or more inhibitors of SOX2 may be administered to the subject in an effective amount. An effective amount of one or more inhibitors of YAP and one or more inhibitors of SOX2 may in some embodiments refer to a quantity sufficient to elicit the biological or medical response that is being sought, including inducing apoptosis of YAP-dependent cancer cells, inhibiting proliferation of YAP-dependent cancer cells, treatment of YAP-dependent cancer, and prevention of YAP-dependent cancer. In some embodiments, an effective amount of one or more inhibitors of SOX2 may refer to a quantity sufficient to reduce the resistance to the effects of a YAP inhibitor or increase the efficacy of a YAP inhibitor to induce apoptosis of YAP-dependent cancer cells, inhibit proliferation of YAP-dependent cancer cells, treat YAP-dependent cancer, or prevent YAP-dependent cancer.

Dosage levels of the one or more inhibitors of YAP and the one or more inhibitors of SOX2 may be varied so as to obtain amounts at the site of the target YAP-dependent cancer cells or the YAP-dependent cancer effective to obtain the desired therapeutic or prophylactic response. Accordingly, the effective amount of the one or more inhibitors of YAP and the one or more inhibitors of SOX2 will depend on the nature and site of the YAP-dependent cancer cells or YAP-dependent cancer, the desired quantity of the one or more inhibitors of YAP and the one or more inhibitors of SOX2 required at the cancer cells or the cancer site to achieve the desired therapeutic or prophylactic response, the nature of the one or more inhibitors of YAP and the one or more inhibitors of SOX2 employed, the route of administration, the physical condition and body size of the subject, among other factors.

An effective amount of the active agent(s) may be presented as different units. For example, an effective amount of the one or more inhibitors of the active agent(s) may presented as a fixed dose, in units of weight of the active agent(s) per body weight of the subject, or in units of weight of the active agent(s) per body area of the subject.

In embodiments of the invention, the active agent(s) may be administered all at once (once-daily dosing), or may be divided and administered more frequently (such as twice-per-day dosing). In some embodiments, the active agent(s) may be administered every other day, or every three days, or every four days, or every five days, or every six days, or once per week, or once per two weeks, or once every three weeks, or once every four weeks, or once every five weeks, or once every six weeks, or once every seven weeks, or once every eight weeks, or once every two months, once every three months, once every four months, once every five months, once every six months, once every seven months, once every eight months, once every nine months, once every ten months, once every eleven months, once every twelve months, once every year, or periods of time therebetween. In some embodiments, the active agent(s) may be administered as a loading dose followed by one or more maintenance doses.

In embodiments of the invention, administration of the active agent(s) may be preceded by a step of identifying the subject in need thereof, i.e., identifying the subject having YAP-dependent cancer, having YAP-dependent cancerous lesions, having YAP-dependent cancer cells, etc. Such identification of the subject may be achieved by methods known in the art for diagnosing the presence of cancer, cancerous lesions, cancerous cells, etc.

The one or more inhibitors of YAP and the one or more inhibitors of SOX2 may contact YAP-dependent cancer cells or may be administered to a subject in a same composition. Alternatively, the one or more inhibitors of YAP and the one or more inhibitors of SOX2 may contact YAP-dependent cancer cells or may be administered to a subject in a different composition.

In some embodiments, the one or more inhibitors of YAP may contact YAP-dependent cancer cells or may be administered to a subject before the one or more inhibitors of SOX2. Or, in certain embodiments, the one or more inhibitors of YAP may contact YAP-dependent cancer cells or may be administered to a subject after the one or more inhibitors of SOX2.

In some embodiments, the one or more inhibitors of YAP may contact YAP-dependent cancer cells or may be administered to a subject shortly before, concurrently, or shortly after, the one or more inhibitors of SOX2. The term “shortly before” as used herein may mean that the one or more inhibitors of YAP contacts YAP-dependent cancer cells or is administered to a subject about 4 hours or less, or about 3 hours or less, or about 2 hours or less, or about 1 hour or less, or about 45 minutes or less, or about 30 minutes or less, or about 15 minutes or less, prior to the one or more inhibitors of SOX2. The term “concurrently” or “concomitantly” (or other forms of these words such as “concurrent” or “concomitant”, respectively) as used herein may mean that the one or more inhibitors of YAP contact YAP-dependent cancer cells or is administered to a subject within about 30 minutes or less, or within about 20 minutes or less, or within about 15 minutes or less, or within about 10 minutes or less, or within about 5 minutes or less, or within about 4 minutes or less, or within about 3 minutes or less, or within about 2 minutes or less, or within about 1 minute or less, or simultaneously, of the one or more inhibitors of SOX2. The term “shortly after” as used herein means that the one or more inhibitors of YAP contact YAP-dependent cancer cells or is administered to a subject about 4 hours or less, or about 3 hours or less, or about 2 hours or less, or about 1 hour or less, or about 45 minutes or less, or about 30 minutes or less, or about 15 minutes or less, after the one or more inhibitors of SOX2.

In embodiments of the invention, contacting the YAP-dependent cancer cells or administering to a subject having YAP-dependent cancer the one or more YAP inhibitors and the one or more SOX2 inhibitors may have an additive effect. The term “additive effect” as used herein means that the effect of contacting the YAP-dependent cancer cells or administering to a subject having YAP-dependent cancer the one or more inhibitors of YAP and the one or more inhibitors of SOX 2 to, for example, induce apoptosis or inhibit proliferation of the cells or treat or prevent YAP-dependent cancer, is approximately equal to the addition of the effects of contacting the cells or administering to the subject the same one or more inhibitors of YAP and the one or more inhibitors of SOX2 by themselves.

In embodiments of the invention, contacting the YAP-dependent cancer cells or administering to a subject having YAP-dependent cancer the one or more YAP inhibitors and the one or more SOX2 inhibitors may have a synergistic effect. The term “synergistic effect” as used herein means that the effect of contacting the YAP-dependent cancer cells or administering to a subject having YAP-dependent cancer the one or more inhibitors of YAP and the one or more inhibitors of SOX 2 to, for example, induce apoptosis or inhibit proliferation of the cells or treat or prevent YAP-dependent cancer, is greater than the addition of the effects of contacting the cells or administering to the subject the same one or more inhibitors of YAP and the one or more inhibitors of SOX2 by themselves. A synergistic effect can be calculated, for example, using suitable models/methods such as the highest single agent model, the Loewe additivity model, the Bliss independence model, the, the Chou-Talalay method, the Sigmoid-Emax equation, or the median-effect equation. Various tools/software can be used to assess synergy, including, but not limited to, CompuSyn, Synergyfinder, Mixlow, COMBIA, MacSynergyII, Combenefit, Combinatorial Drug Assembler (http://cda.i-pharm.org/), Synergy Maps (http://richlewis42.github.io/synergy-maps/), DT-Web (http://alpha.dmi.unict.it/dtweb/), and TIMMA-R.

In some embodiments, the methods and uses of the present invention may further comprise administering one or more inhibitors of Myc. Such inhibitors may be used to contact cells or may be administered shortly before, shortly after, or concurrently, with the one or more inhibitors of YAP and the one or more inhibitors of SOX2, or with the one of more inhibitors of SOX2. Myc inhibition may be achieved by indirect Myc suppression such as via inhibition of regulators of Myc protein stability, inhibition of pathways that are involved in Myc translation, or inhibition of Myc chromatin remodeling; or by small molecules that directly block Myc interaction with Myc associated factor X (Max, to which Myc must dimerize to function) or that block binding of Myc-Max to DNA. Examples of MYC inhibitors may include, but are not limited to, JQ1, ZEN-3694, OTX015, TEN-010, 17-AAG, 17-DMAG, alisertib, IIA6B17, 10058-F4, 10074-G5, 10074-A4, JY-3-094, 3jc48-3, Mycro3, KJ-Pyr-9, sAJM589, MYCMI-6, MYRA-A, NSC308848, JKY-2-169, and KSI-3716, KSI-2826, FBN-1503, KSI-1449, KSI-2303, APTO-253, MYCi975 (NUCC-0200975), lusianthridin, MYCi361 (NUCC-0196361), ML327, IZCZ-3, CMLD010509 (SDS-1-021), and stauprimide.

### Kits Comprising Pharmaceutical Compositions and a Package Insert

An aspect of the invention relates to kits containing one or more inhibitors of YAP and one or more inhibitors SOX2, either in the same pharmaceutical composition or different pharmaceutical compositions, and a package insert. As used herein, a “kit” is a commercial unit of sale, which may comprise a fixed number of doses of the one or more pharmaceutical compositions. By way of example only, a kit may provide a 30-day supply of dosage units of one or more fixed strengths, the kit comprising 30 dosage units, 60 dosage units, 90 dosage units, 120 dosage units, or other appropriate number according to a physician's instruction. As another example, a kit may provide a 90-day supply of dosage units.

In some embodiments, the kit may comprise a pharmaceutical composition comprising one or more inhibitors of SOX2 according to the present invention, and a package insert.

As used herein, “package insert” means a document which provides information on the use of the one or more pharmaceutical compositions, safety information, and other information required by a regulatory agency. A package insert can be a physical printed document in some embodiments. Alternatively, a package insert can be made available electronically to the user, such as via the Daily Med service of the National Library of Medicines of the National Institute of Health, which provides up-to-date prescribing information. (See https://dailymed.nlm.nih.gov/dailymed/index.cfm.)

In some embodiments, the package insert may inform a user of the kit that the pharmaceutical composition(s) may be administered according to the methods of use of the present invention.

### EXAMPLES

### Example 1

The following example describes a study on the role of YAP in KRAS mutant pancreatic tumors (see also Murakami et al. 2019, which is incorporated herein by reference).

### Ablation of YAP Induced Tumor Regression and Prolongs Survival in Mice Bearing Established KRAS Mutant Pancreatic Tumors.

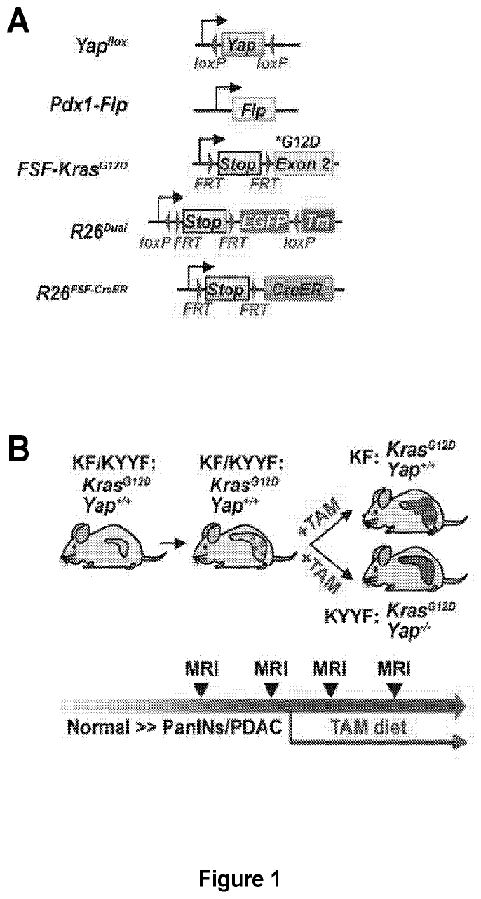

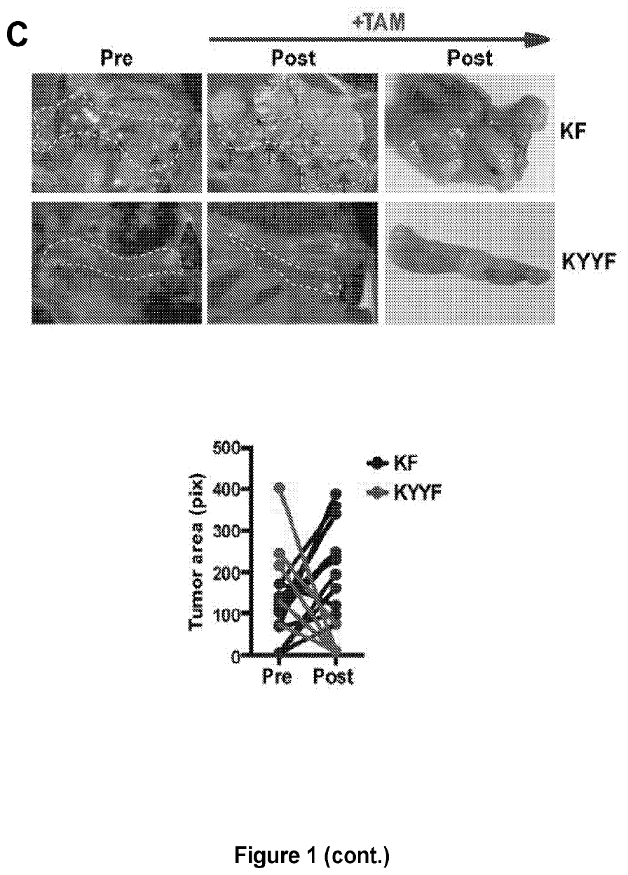

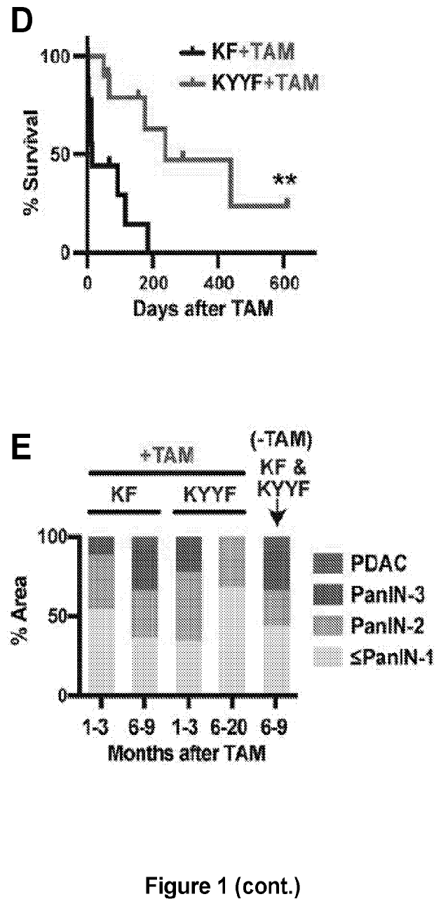

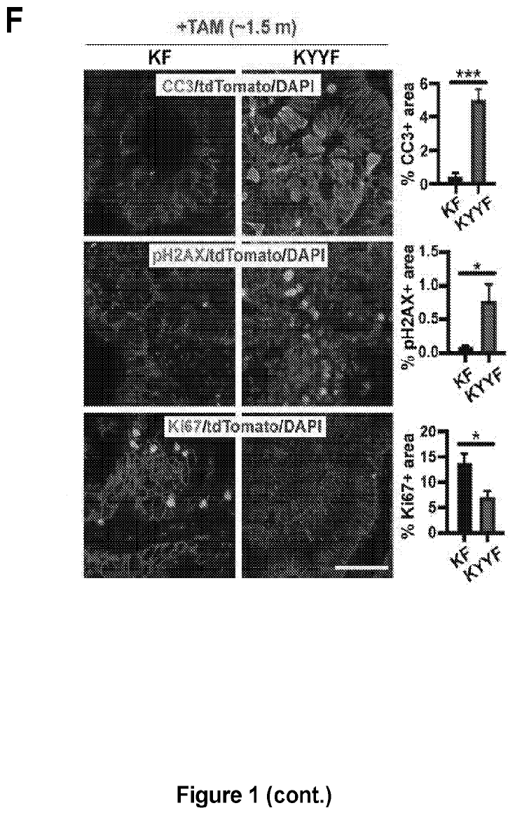

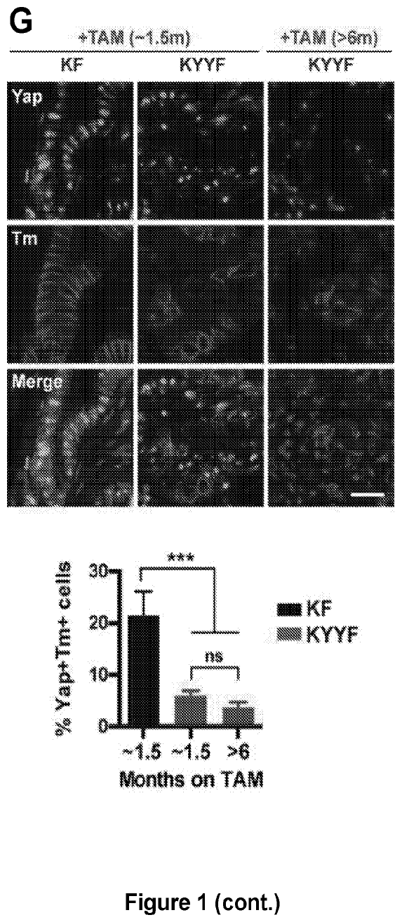

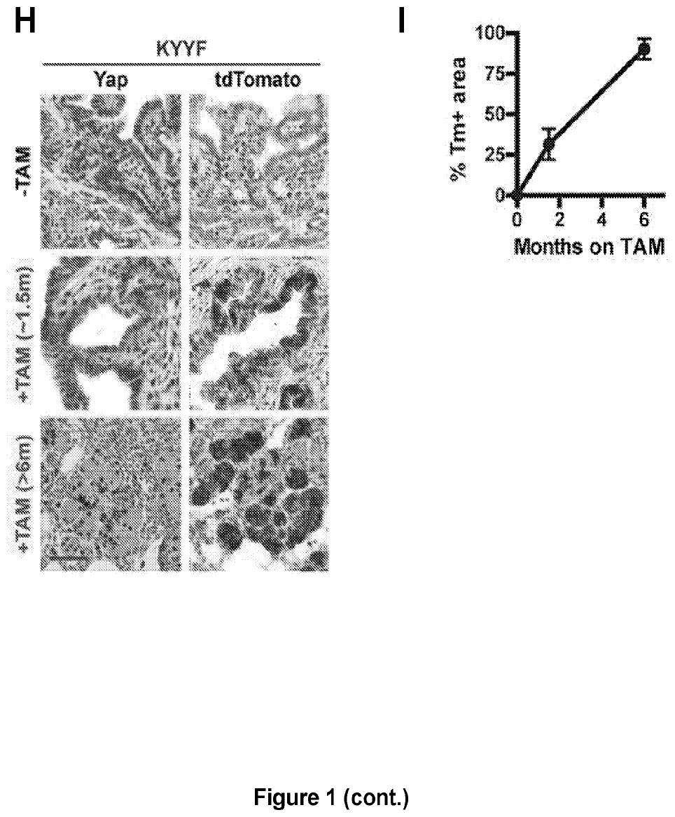

An inducible genetically engineered mouse model (GEMM) was developed that combined the Flp-FRT and Cre-loxP recombination systems, which allowed YAP to be switched off from spontaneously developed KRAS mutant pancreatic tumors in immune competent mice (FIGS. 1A-B). The GEMM also incorporated a dual-fluorescent reporter (R26^{dual}), which marked the tumor cells according to their mutational statuses so that tumor cells could be distinguished from stromal cells and unrecombined normal tissues, and so that YAP competent tumor cells could be distinguished from YAP deficient tumor cells (FIGS. 1A and 1B). Two cohorts—KF (FSF-KRAS^{G12D/+}; R26^{FSF-CreER/Dual}; YAP^{+/+}; Pdx1-Flp) and KYYF (FSF-KRAS^{G12D/+}; R26^{FSF-CreER/Dual}; YAP^{flox/flox}; Pdx1-Flp)—were subjected to detailed analysis. In both cohorts, Flp-recombinase directed by the Pdx1 promoter (Pdx1-Flp) removed the FRT-flanked STOP cassettes from the FSF-Kras^{G12D}, R26^{dual}; and R26^{FSF-CreER} alleles in pancreatic progenitor cells, which resulted in the expression of KRAS^{G12D}, EGFP and latent CreER throughout the pancreatic parenchyma (FIGS. 1A and 1B) (Schönhuber et al. 2014). Without Tamoxifen (TAM) treatment, CreER remained inactive and could not induce recombination in the YAP^{flox/flox} alleles, and therefore YAP expression was maintained in both KF and KYYF mice (FIGS. 1A and 1B).

MRI was used to monitor disease progression over time. Upon detection of multiple frank lesions via MRI, mice were switched to a TAM-containing diet to activate CreER, which induced LoxP-mediated recombination at the YAP^{flox/flox} and R26^{dual} alleles, resulting in simultaneous deletion of YAP and EGFP and activation of tdTomato (Tm) in the KRAS^{G12D} expressing pancreatic neoplastic epithelial cells of KYYF mice (FIGS. 1A, 1B and 1G). In contrast, YAP remained expressed in Tm⁺ tumor cells in KF mice (FIGS. 1B and 1G). As indicated by Tm and YAP staining, TAM-induced recombination is mosaic with the percentage of recombined (Tm⁺YAP⁻) cells gradually increased after extended treatment (FIGS. 1H and 1I). Despite the slow recombination rate, sequential MRI imaging indicated shrinkage of established pancreatic lesions (some to undetectable levels) in KYYF mice after three months of TAM treatment (FIG. 1C). In contrast, existing lesions continued to grow while new nodules appearing in KF mice over the same time period under TAM treatment (FIG. 1C). Consistent with findings from MRI, KYYF mice exhibited significantly prolonged survival compared to KF mice following TAM treatment (FIG. 1D), which correlated with gradual decrease in advanced lesions (FIG. 1E). These results demonstrate the requirement for YAP in maintenance of KRAS mutant PDAC tumors.

### YAP Ablation Triggered Growth Arrest and Apoptosis in KRAS Mutant Pancreatic Tumor Cells In Vitro and In Vivo.

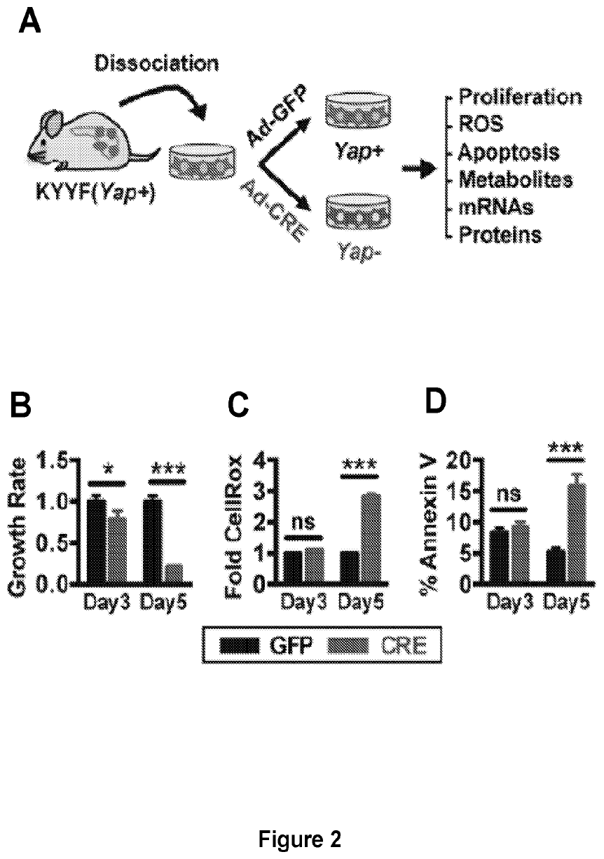

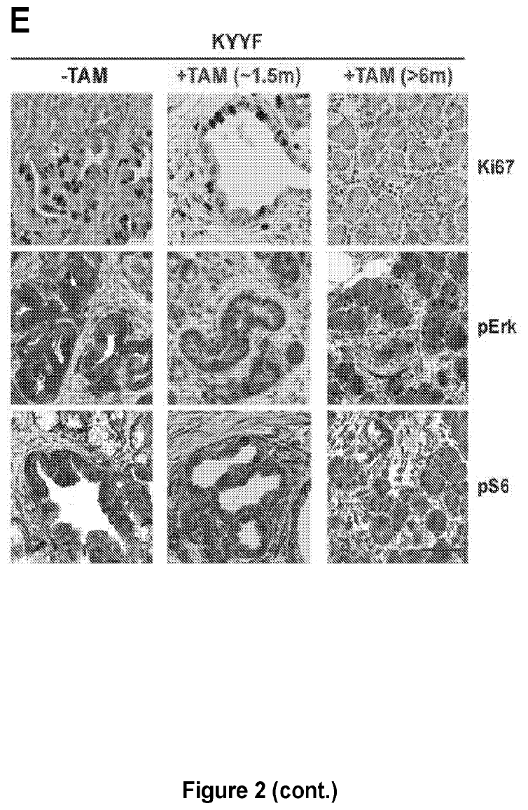

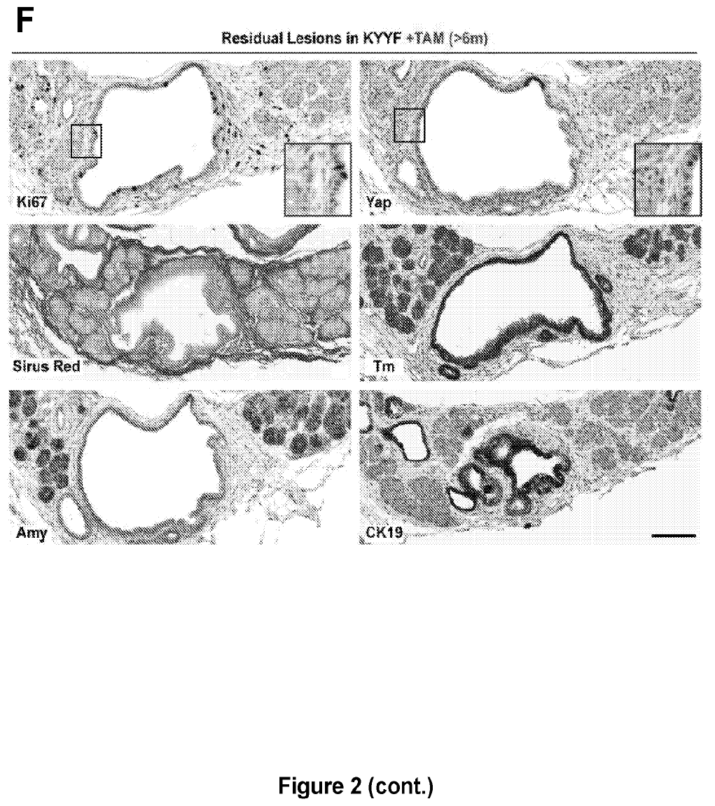

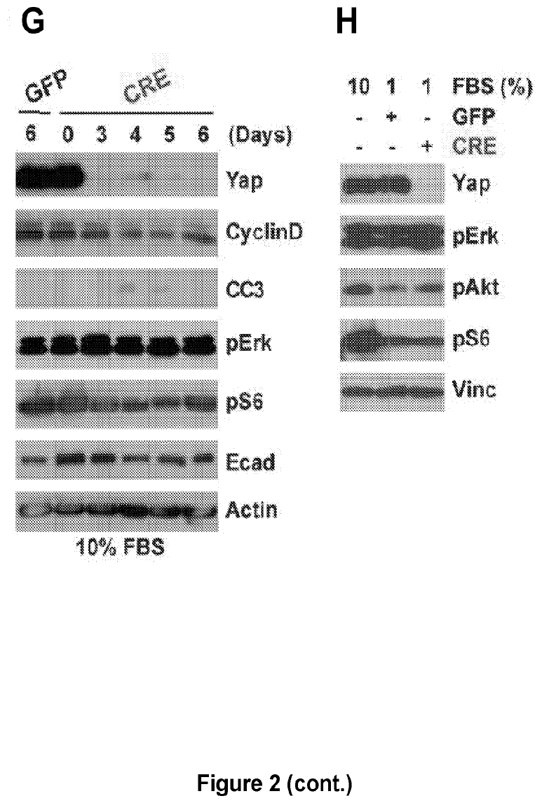

Corresponding to the shrinkage of frank lesions, the percentage of Cleaved-Caspase 3 (CC3) positive apoptotic or pH2AX positive stressed cells increased dramatically within the Tm⁺YAP⁻ population of KYYF pancreata but not in KF pancreata after 1.5 months of TAM treatment (FIGS. 1F and 1G). Conversely, the percentage of Ki67⁺ proliferating cells decreased significantly in KYYF relative to KF pancreata (FIGS. 1F and 2E). After extended TAM treatment, the vast majority of KYYF pancreata became completely quiescent except for a few remnant Tm⁺YAP⁺ ductal lesions that failed to undergo complete recombination (FIGS. 2E and 2F). The quiescent Tm⁺YAP⁻KYYF pancreatic cells remained positive for pERK and pS6 (FIG. 2E), suggesting that YAP was not required for sustaining MAPK and mTOR signaling.

Primary culture from an invasive pancreatic tumor isolated from a KYYF mouse that did not undergo TAM treatment was established, and YAP knockout was induced in these cells by infecting them with Ad-CRE or Ad-GFP as control (FIG. 2A). While Ad-GFP treatment had no effect on YAP expression, cell proliferation, or survival, Ad-CRE reduced cell proliferation associated with loss of YAP from 3 days post treatment, followed by a delayed buildup of cellular ROS and apoptotic makers (FIGS. 2B-2D). YAP loss in vitro also had little effect on pERK and pS6 levels in either low or high serum condition (FIGS. 2G and 2H), confirming that YAP controls the growth and survival of KRAS mutant pancreatic tumor cells through mechanisms independent of the MAPK and mTOR pathways.

### YAP Partnered With the TEAD Family of Transcription Factors to Directly Transcribe the Myc Gene to Sustain Nucleotide Synthesis In Vitro and In Vivo.

Because it was previously shown that the YAP/TEAD transcriptional complex cooperates in cis with Myc in promoting the transcription of genes important for growth and proliferation (Croci et al. 2017), it was investigated whether YAP functions through or in collaboration with Myc in maintaining the expression of the metabolic genes necessary for the survival of KRAS mutant pancreatic tumor cells.

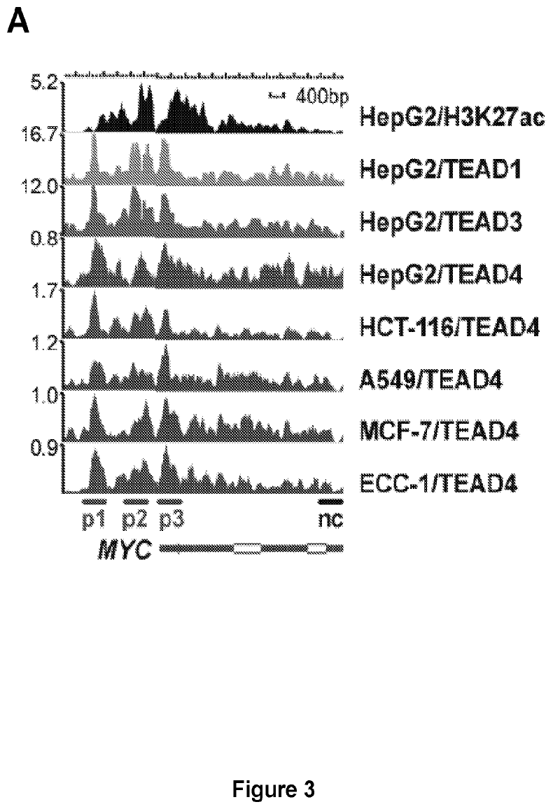

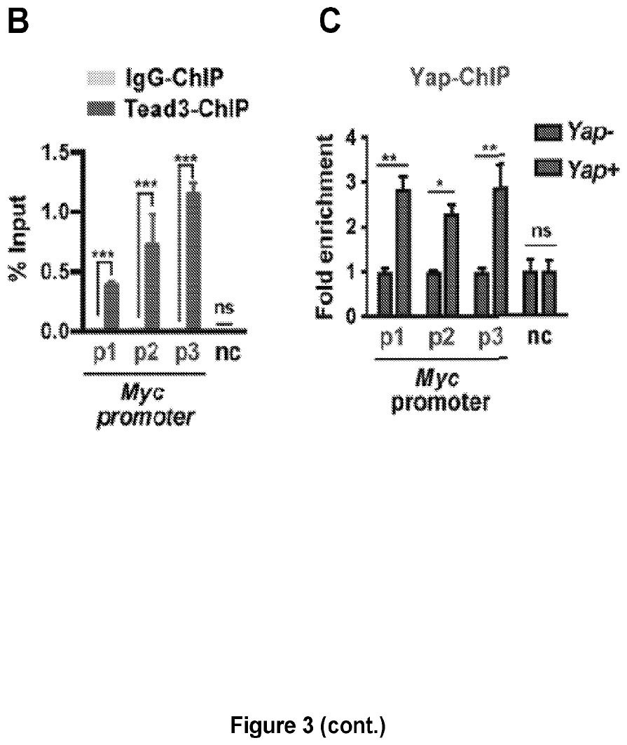

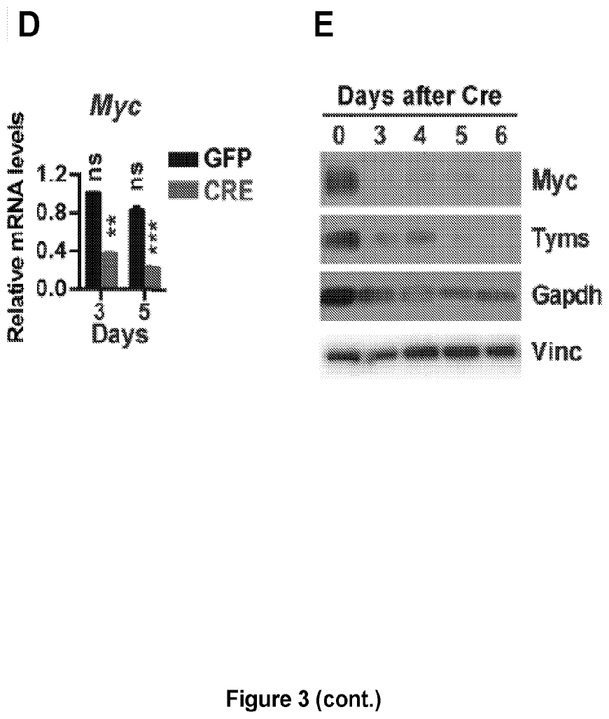

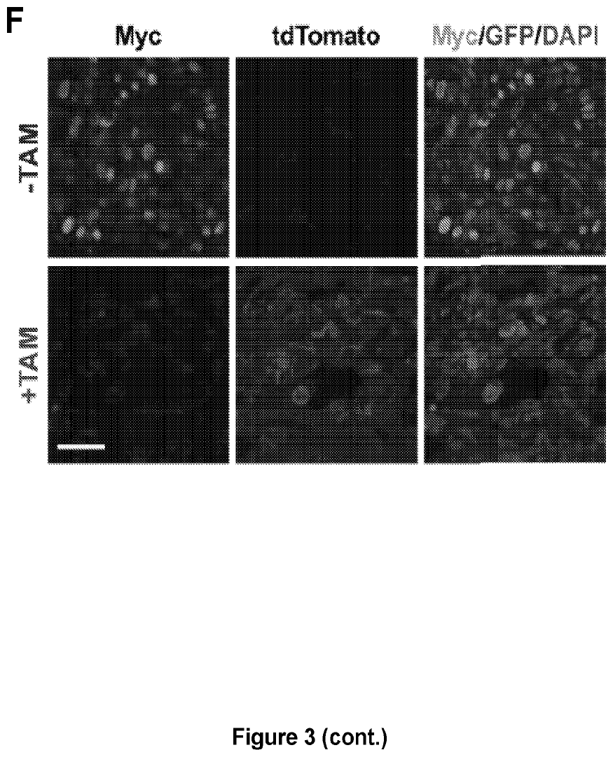

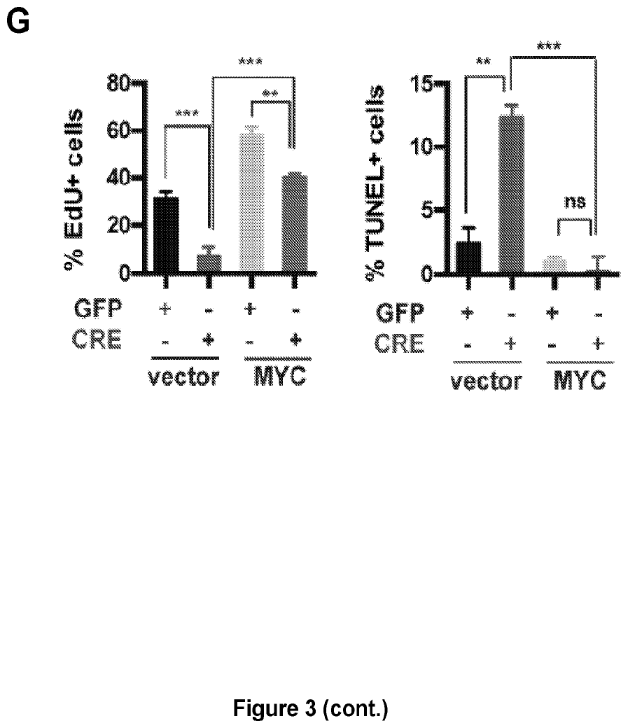

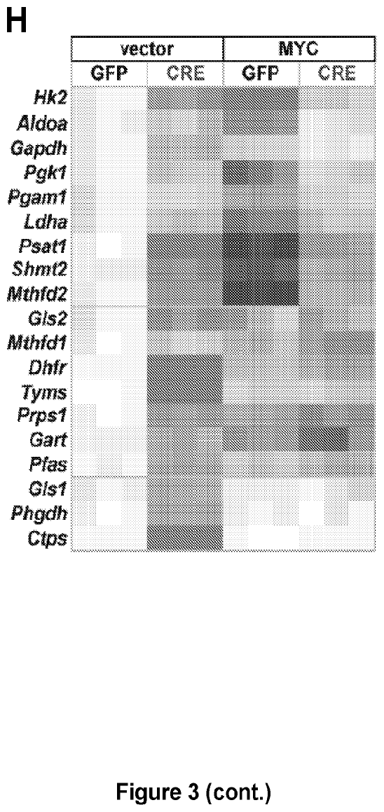

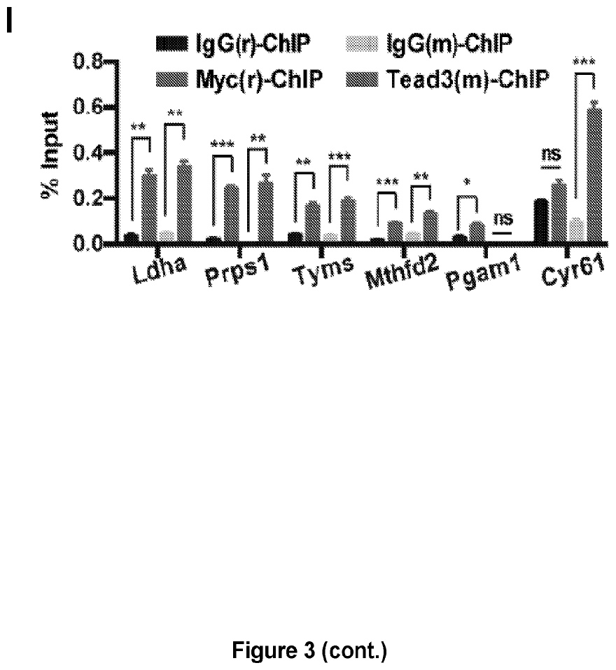

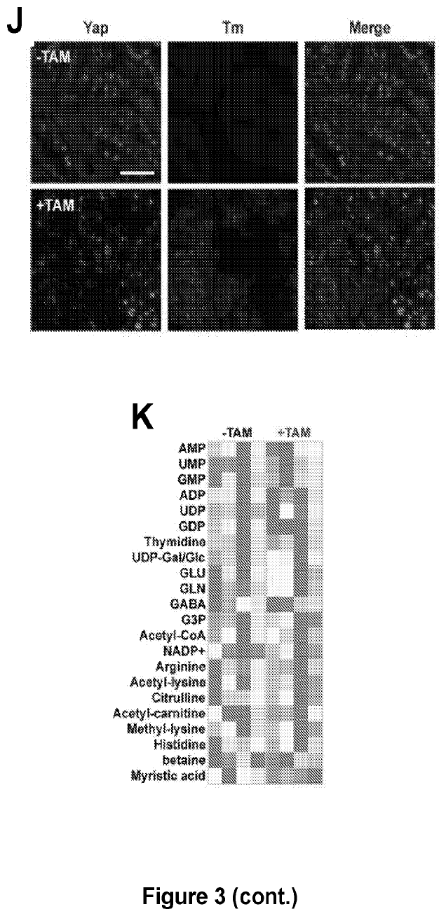

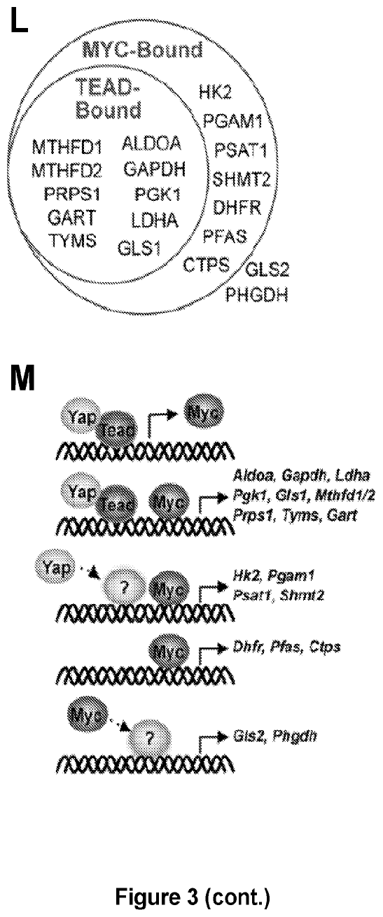

The proximal MYC promoter was examined for possible binding by TEAD, and it was found that TEAD1, TEAD3, and TEAD4 consistently bind across multiple cancer cell lines at three major sites (p1-3) along an approximately 4 kb span from the transcription start site of the MYC gene, which overlap with the H3K27Ac active transcription marks (FIG. 3A). Chromatin immunoprecipitation (ChIP) was used to confirm that in primary murine pancreatic tumor cells, TEAD3 binds to three conserved regions of the mouse Myc promoter corresponding to the TEAD-binding peaks in human cells, but not at the 3′ UTR (FIG. 3B). YAP ChIP was performed in YAP⁺ or YAP⁻ pancreatic tumor cells, which showed specific enrichment of YAP antibody to the three TEAD-binding sites in YAP⁺ but not YAP⁻ cells (FIG. 3C). Further, qRT-PCR, western blot, and IF analyses showed that ablation of YAP or TEAD from KRAS mutant pancreatic tumor cells reduced both the mRNA and protein levels of Myc in vitro and in vivo (FIGS. 3D-3F, 3J, and 3K; Table 1), demonstrating that the YAP/TEAD transcriptional complex directly promotes the expression of Myc.

TABLE 1
P-value ranking of metabolic pathways significantly downregulated
in TAM-treated versus untreated orthotopic pancreatic tumors
in targeted LC-MS/MS metabolic analysis (n = 4).

| Pathways Downregulated in TAM-Treated Tumors | P value |
|---|---|
| Nitrogen metabolism | 2.23E−04 |
| Arginine and proline metabolism | 3.22E−04 |
| Purine metabolism | 2.45E−03 |
| Pyrimidine metabolism | 2.53E−03 |
| Butanoate metabolism | 3.61E−03 |

### YAP and Myc Cooperated at Multiple Levels to Maintain the Expression of Metabolic Genes That are Important for Pancreatic Tumor Cell Proliferation and Survival.

To determine how downregulation of Myc contributes to the phenotypes induced by YAP loss, KYYF lines were generated that stably expressed exogenous human MYC or vector control, and were treated with either Ad-GFP or Ad-CRE. Overexpression of either MYC prevented apoptosis and cell cycle arrest induced by YAP ablation (FIG. 3G), confirming the inhibition of Myc as the major cause of cell death and growth arrest following YAP loss in KRAS mutant pancreatic tumor cells.

Even though MYC overexpression rescued the growth and survival of YAP-deleted PDAC, it did not fully over-write the inhibitory effects of YAP ablation on cell proliferation, as indicated by the significant reduced growth rates of CRE-treated relative to GFP-treated MYC overexpressing cells (FIG. 3G). Correspondingly, while MYC overexpression rescued the expression of all the metabolic genes that were downregulated in control cells following YAP ablation, nearly half of those genes were expressed at significantly lower levels in CRE-treated versus GFP-treated MYC-overexpressing cells (FIG. 3H), indicating that they are likely subjected to additional regulation by YAP independent of Myc.

To assess the functional hierarchy between the YAP/TEAD transcriptional complex and Myc in regulating metabolic genes, the occupancies of TEAD4 and MYC on the proximal promoters of YAP-regulated metabolic genes were compared. In all the cell lines examined clear, TEAD4 was enriched at the active transcription sites (as indicated by H3K27Ac) of over half of these genes, all of which also exhibited robust MYC binding at overlapping or adjacent sites (FIG. 3L). ChIP analysis confirmed that Myc and TEAD also co-occupied the promoters of Ldha, Prps1, Tyms and Mthfd2 in primary murine pancreatic tumor cells (FIG. 3I). In contrast, the Pgam1 promoter was bound by Myc but not by TEAD3, whereas the canonical TEAD target Cyr61 showed strong enrichment for TEAD3 but not Myc (FIG. 3I). Thus, the YAP/TEAD transcription complex may function either in conjunction with or through Myc to regulate the transcription of metabolic genes (FIG. 3M).

### Upregulation of SOX2 Compensated for YAP Loss, Restoring Myc Expression, Metabolic Homeostasis, and Survival in a Subset of YAP Deficient Pancreatic Tumor Cells.

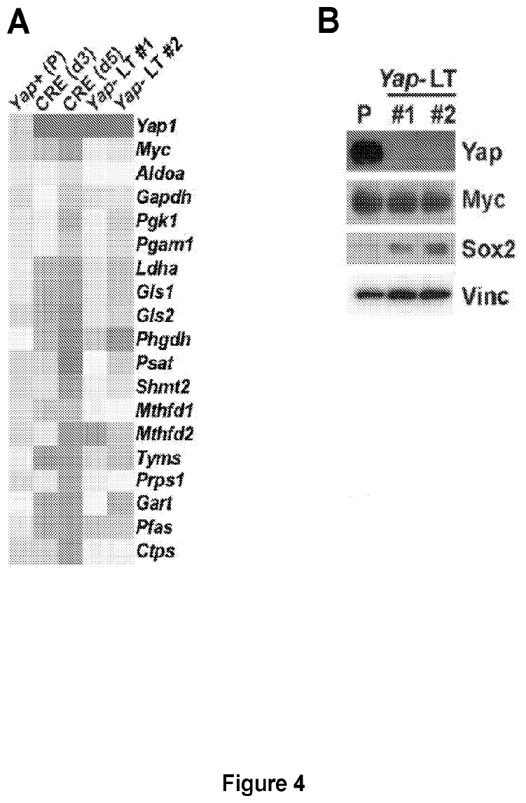

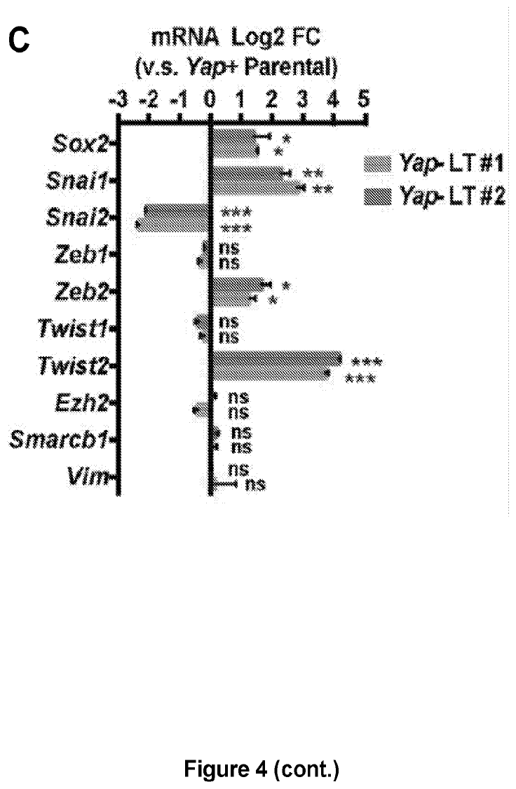

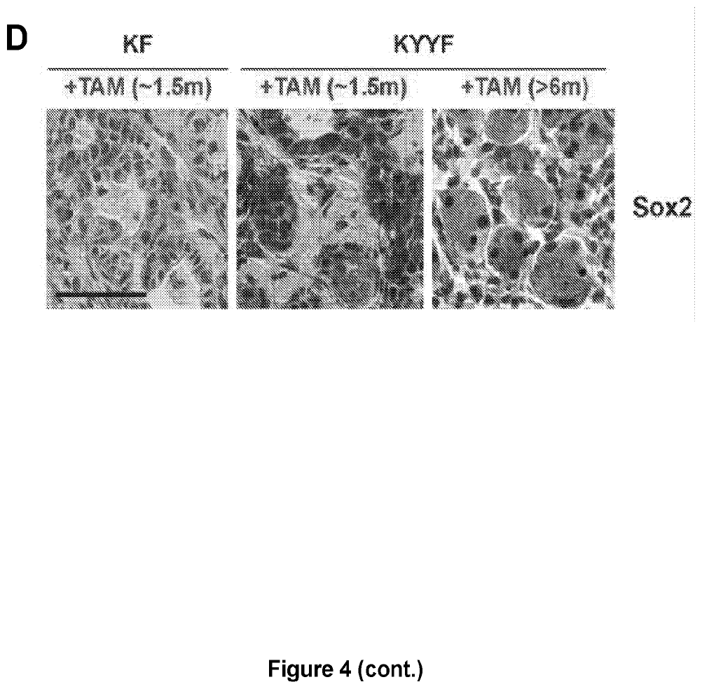

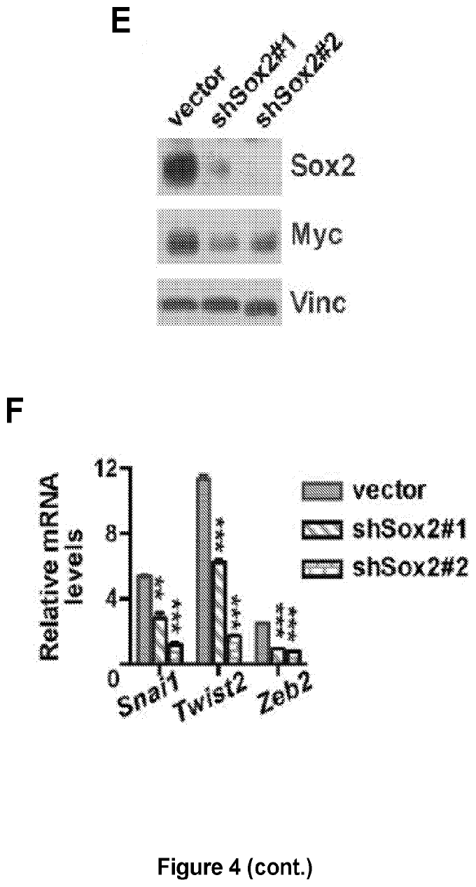

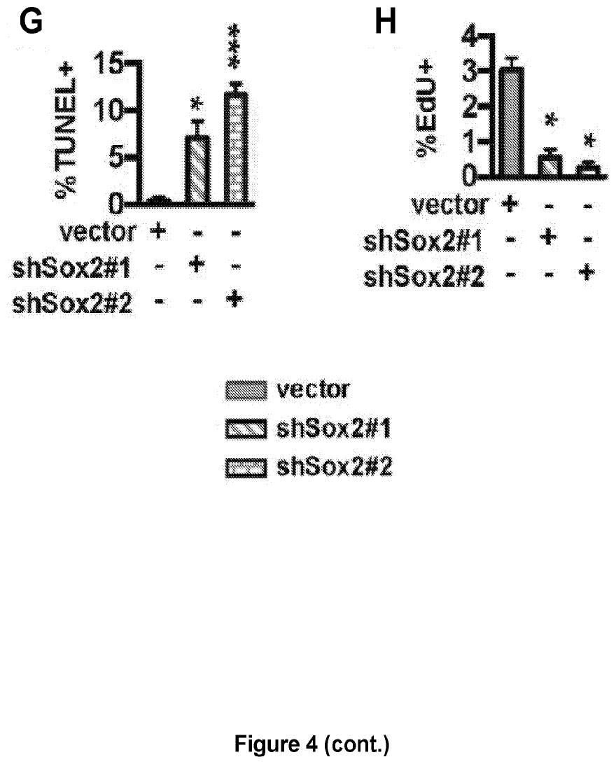

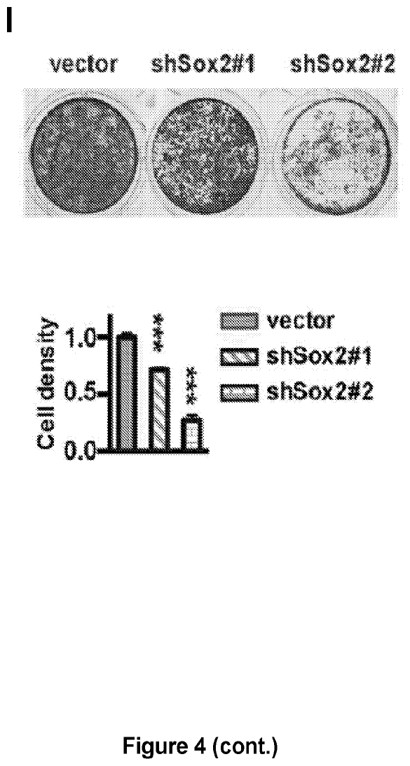

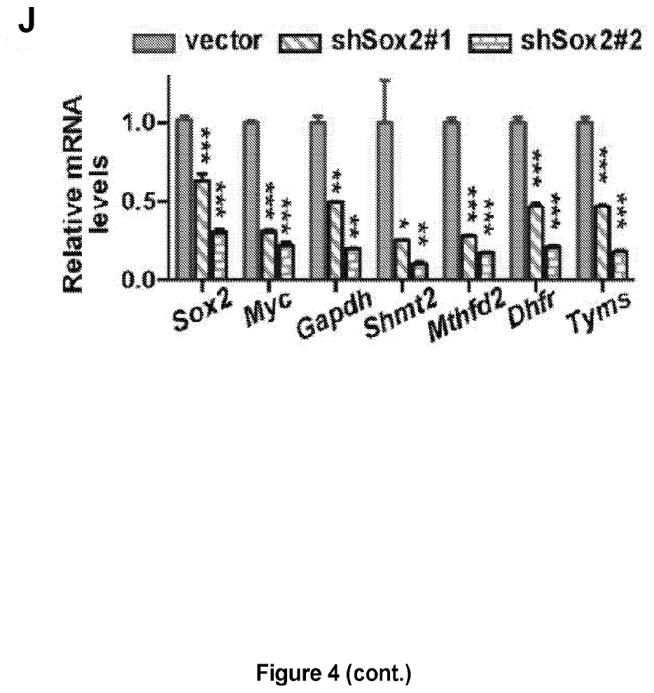

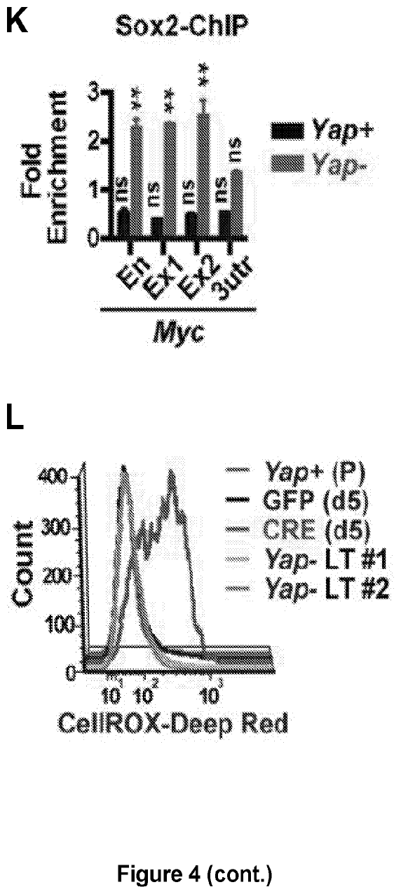

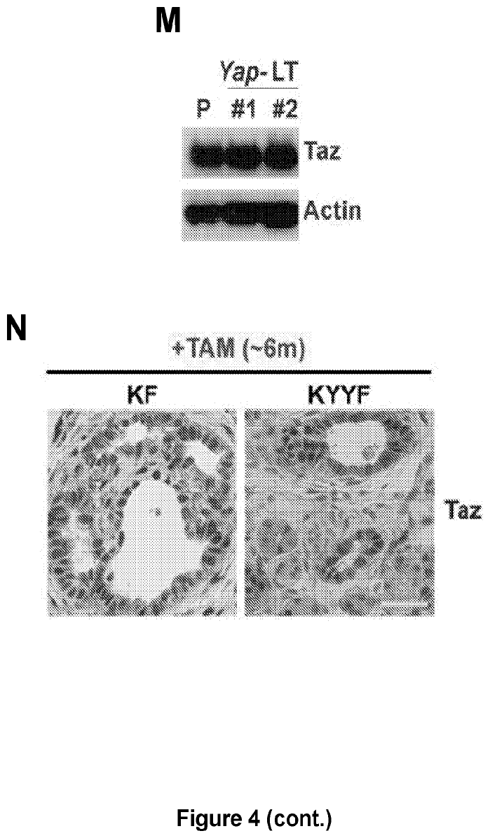

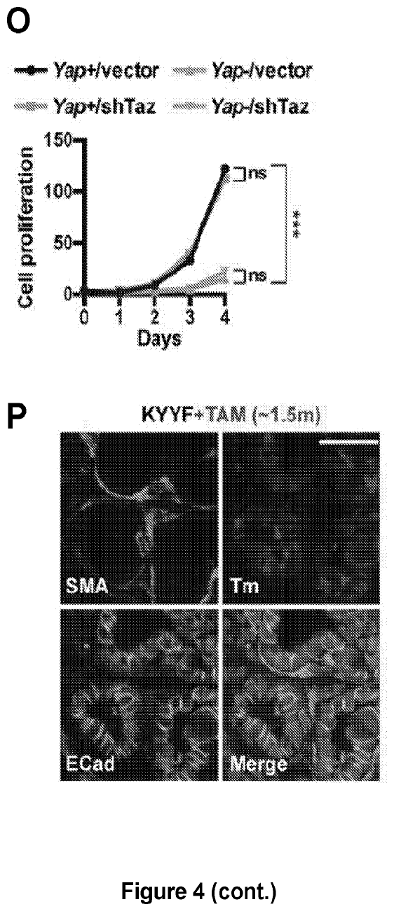

Despite the cell death and growth arrest induced by YAP ablation (FIGS. 2B-2D), a significant fraction of KRAS mutant pancreatic tumor cells survived long term YAP loss, and over time regained ROS homeostasis (FIG. 4L). This revival coincided with recovery in the expression of Myc and many of the metabolic enzymes downregulated following acute YAP loss (FIGS. 4A and 4B), which suggests the existence of compensatory mechanism(s) that allow long-term (LT) surviving YAP⁻ pancreatic tumor cells to restore Myc expression and Myc-controlled metabolic programs.

TAZ has been shown to be upregulated in response to YAP loss and compensate for its function (Moroishi et al., 2015). However, no increase in TAZ expression following YAP ablation was observed in vitro or in vivo (FIGS. 4M and 4N). Knockdown of TAZ also did not significantly impact the growth of pancreatic tumor cells in the presence or absence of YAP (FIG. 4O), suggesting that TAZ cannot functionally replace YAP in sustaining pancreatic tumor growth.

Overexpression of YAP has been previously shown to allow KRAS mutant colon cancer cell line HCT-116 to survive KRAS silencing by upregulating the epithelial-mesenchymal transition (EMT) program (Shao et al., 2014). With this background, the expression of EMT-related genes was compared in YAP⁺ and YAP⁻ LT-surviving pancreatic tumor cells. A number of EMT genes including SOX2, Snail, Zeb2, and TWIST2 were significantly upregulated in YAP⁻ cells compared to YAP⁺ cells, whereas Snail was significantly downregulated (FIGS. 4B and 4C). The upregulation of SOX2 was also apparent in vivo in KYYF pancreata relative to KF pancreata after ˜1.5 months of TAM treatment (FIG. 4D). Despite the upregulation of several EMT genes, YAP⁻ cells did not upregulate Vim or Zeb1—two most widely accepted mesenchymal markers (FIG. 4C). Tm⁺Yap⁻KRAS mutant neoplastic cells also maintained E-Cad expression and epithelial morphology in vivo (FIG. 4P). These data suggest that YAP loss induces a partial but not overt EMT program in KRAS mutant pancreatic tumor cells.

Because SOX2 has been previously shown to promote EMT and stemness in many types of tumor cells including human PDAC cells (Herreros-Villanueva et al. 2013; Wuebben and Rizzino 2017), it was investigated whether the upregulation of SOX2 could be responsible for inducing the partial EMT program and allowing pancreatic tumor cells to survive YAP ablation. Knockdown of SOX2 with two independent shRNAs caused dose-dependent downregulation of Snai1, TWIST2 and Zeb2 and induction of cell death and growth arrest in YAP null pancreatic tumor cells, which corresponded to significant reduction in the expression of Myc and Myc-regulated metabolic genes (FIGS. 4E-4J). Further, ChIP-qPCR was used to confirm that SOX2 specifically binds to a previously reported enhancer region and exons 1 and 2 but not the 3′-UTR of the Myc gene in YAP null pancreatic tumor cells (FIG. 4K). These results suggest that, surprisingly, upregulation of SOX2 could compensate for YAP loss to rescue Myc expression and metabolic homeostasis, allowing pancreatic tumor cells to survive YAP ablation.

### Metabolic-Crisis-Triggered Epigenetic Reprogramming Drove SOX2 Upregulation and Lineage Shift Following YAP Ablation in Pancreatic Tumor Cells.

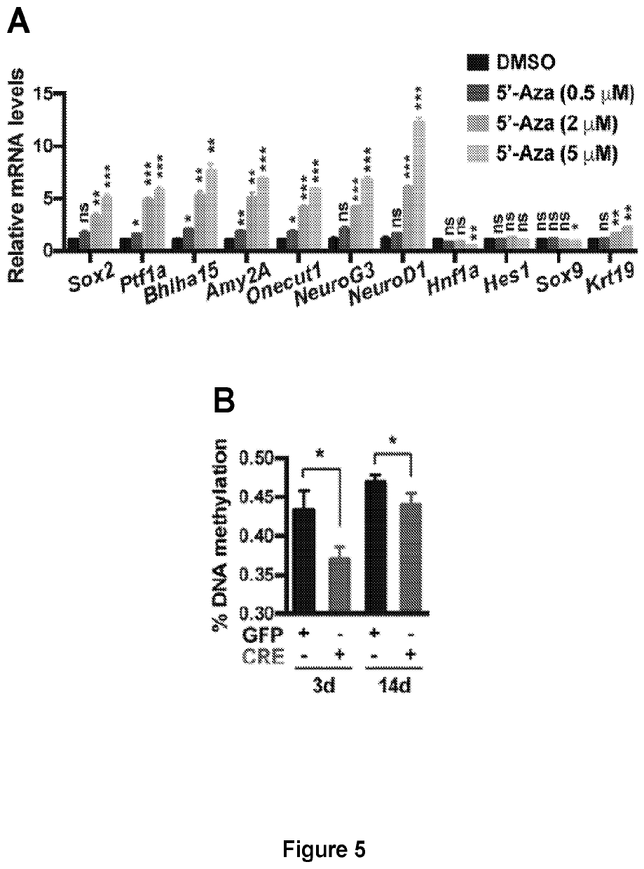

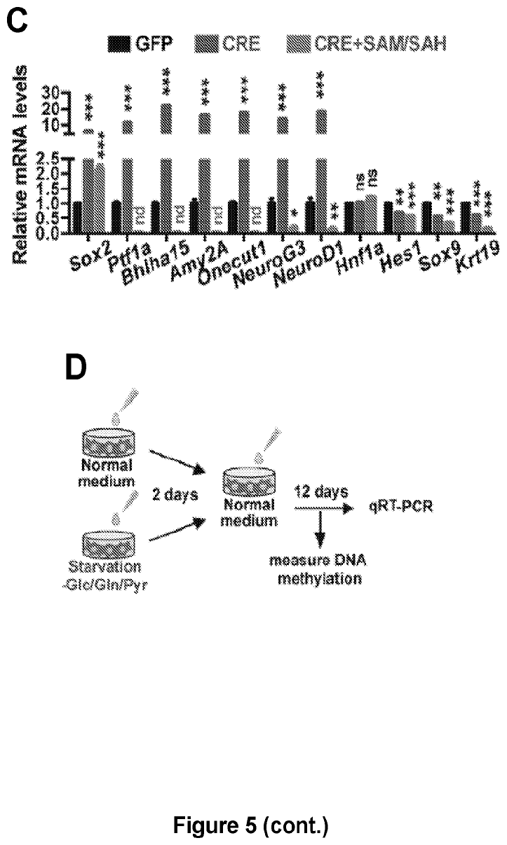

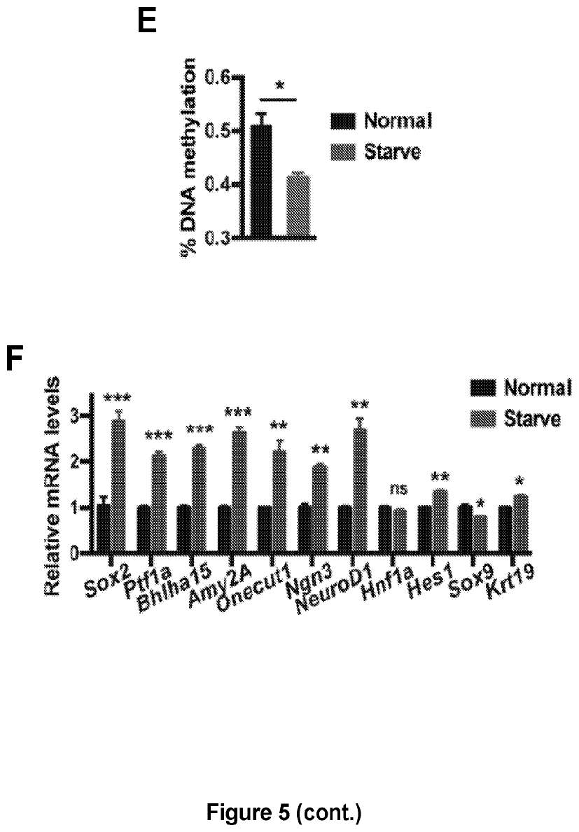

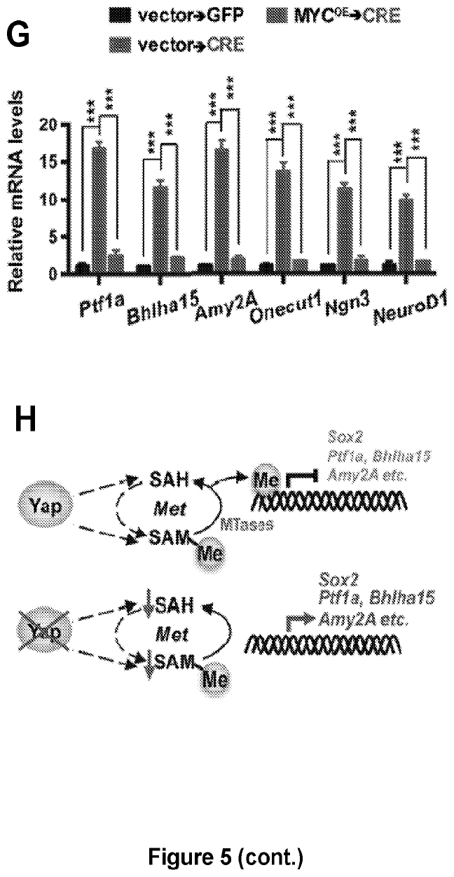

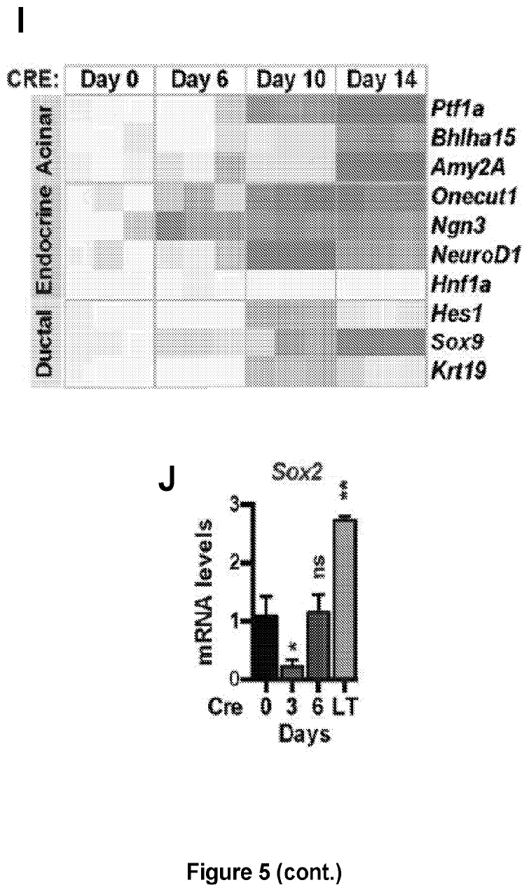

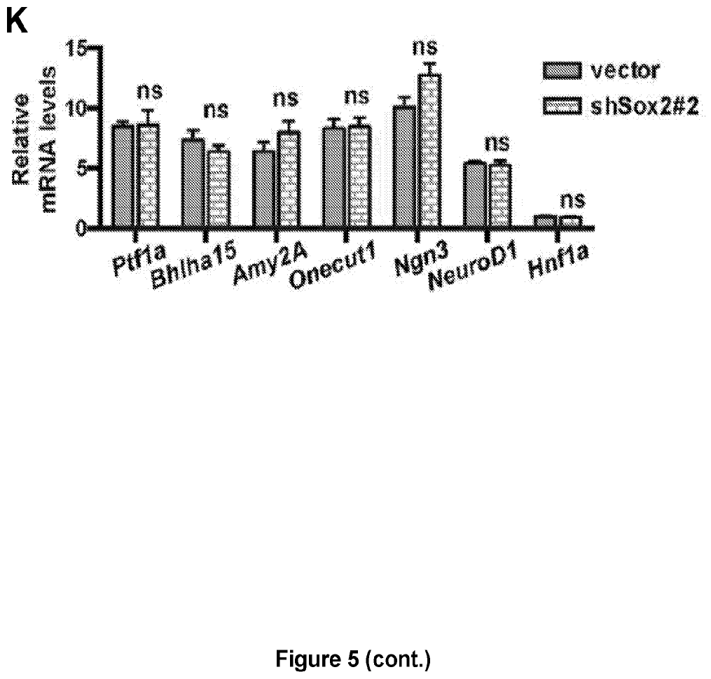

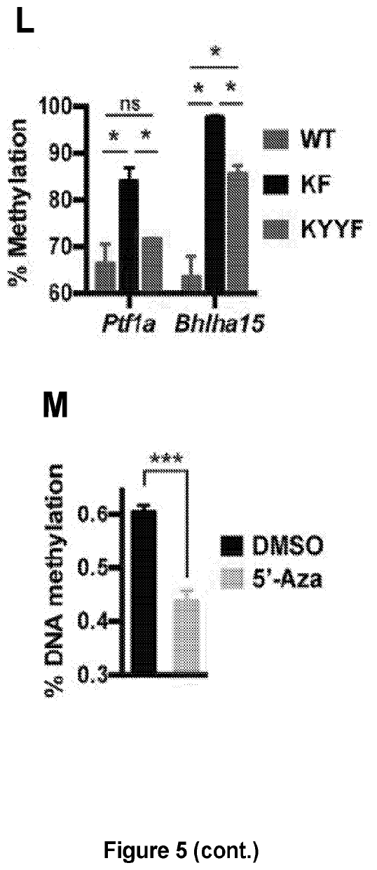

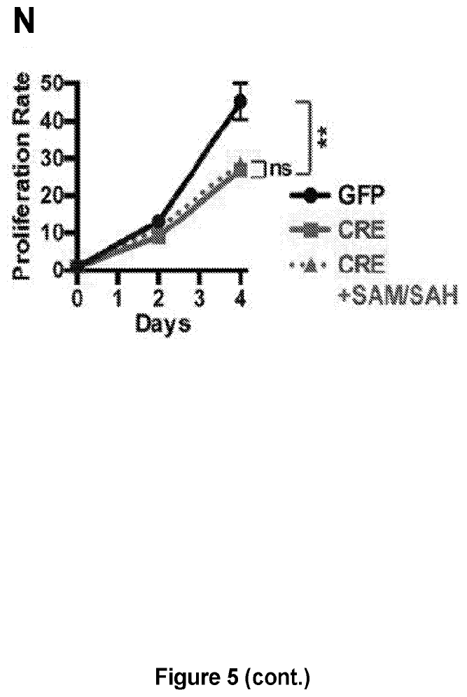

In contrast to the rapid decrease in the expression of Myc and metabolic genes following YAP ablation (FIG. 4A), the changes in the expression of lineage markers occurred slowly, and did not peak until more than one week after YAP was first deleted (FIG. 5I). Even though the kinetics of the lineage shift closely followed that of SOX2 upregulation (FIG. 5J), SOX2 knockdown had very little effects on the expression of these genes in YAP⁻ cells in vitro (FIG. 5K), and SOX2 expression eventually became barely detectable in regenerated acinar-like cells in KYYF pancreata after >6 months of TAM treatment (FIG. 4D). These results suggest while SOX2 plays a critical role in rescuing Myc expression and cell survival upon YAP loss, it does not drive the re-expression of acinar lineage genes.

It was investigated whether DNA demethylation could be responsible for reactivating pancreatic lineage genes following YAP loss, as DNA-methylation-mediated gene silencing is critical for the establishment and maintenance of lineage commitment and cellular identity (Suelves et al., 2016), and DNA methylation requires methyl groups to be donated through conversion of S-Adenosyl methionine (SAM) to S-Adenosyl homocysteine (SAH), both of which were significantly downregulated in response to YAP ablation in primary pancreatic tumor cells (FIG. 2E).

Quantitative methylation-specific PCR (qMSP) was used to confirm that the promoters of acinar lineage genes Ptf1a and Bhlha15 became heavily methylated in KF pancreatic tumors in contrast to wild type (WT) pancreata, which was partially reversed in KYYF pancreata after TAM treatment (FIG. 5L). To directly assess the effects of DNA de-methylation on the expression of pancreatic lineage genes, YAP⁺ primary pancreatic tumor cells were treated with vehicle control or DNA methylation inhibitor 5-azacytidine (5-Aza). Along with global DNA de-methylation (FIG. 5M), 5-Aza treatment induced dose-dependent upregulation acinar and endocrine lineage genes as well as SOX2 (FIG. 5A), demonstrating that DNA methylation is at least partially responsible for silencing these genes in YAP⁺ pancreatic tumor cells. The levels of global DNA methylation in primary KYYF pancreatic tumor cells were measured following treatment with Ad-GFP or Ad-CRE, and it was confirmed that YAP ablation induced a rapid and significant decline in global DNA methylation, which partially recovered over time (FIG. 5B). Given that the global DNA de-methylation coincided with the drop in SAM and SAH levels following YAP deletion (FIG. 2E), it was tested whether supplementing the growth medium with SAM/SAH after CRE treatment could prevent the reactivation of pancreatic lineage genes. Addition of exogenous SAM/SAH, which did not prevent the proliferation decrease in CRE-treated KYYF cells (FIG. 5N), caused complete or near complete silencing of pancreatic lineage genes including Ptf1a, Bhlha15, Amy2A, Onecut1, NeuroG3, and NeuroD1 and strongly suppressed SOX2 upregulation, but had no effect on the expression of Hnf1a (FIG. 5C). SAM/SAH also caused downregulation in ductal markers Hes1, Sox9 and Krt19 to various degrees, suggesting that these genes may also subjected to some degree of regulation by methylation (FIG. 5C).

To confirm that the metabolic stress was the trigger of global DNA de-methylation and lineage shift in PDAC cells following YAP loss, YAP⁺ pancreatic tumor cells were starved for two days in growth media deprived of Glc, Gln, and Pyr, followed by recover in normal growth media for addition 12 days (FIG. 5D). As shown in FIGS. 5E and 5F, temporary deprivation of carbon sources induced global demethylation accompanied by upregulation of SOX2 and acinar and endocrine lineage markers in YAP⁺ pancreatic tumor cells, recapitulating the effects of 5-Aza treatment (FIGS. 5A and 5N). Moreover, overexpression of MYC was confirmed, which prevented the short-term growth arrest and cell death induced by CRE treatment (FIG. 3G), either completely or partially suppressed the acquisition of acinar and endocrine genes at 14 days after virus infection (FIG. 5G).

Together, the data support a model in which YAP ablation from pancreatic tumor cells causes acute metabolic crisis, which triggers not only cell cycle arrest and apoptosis but also DNA de-methylation and epigenetic reprogramming, resulting in SOX2 upregulation that restores Myc expression and metabolic homeostasis, and de-repression of acinar lineage genes that gradually convert the surviving Yap-deficient neoplastic ductal cells into acinar-like cells (FIG. 5H).

### Example 2

The following example describes a study on how other inhibitors may impact YAP loss in PDAC cells.

### BET Inhibitors Blocked PDAC Cells From Adapting to YAP Loss.

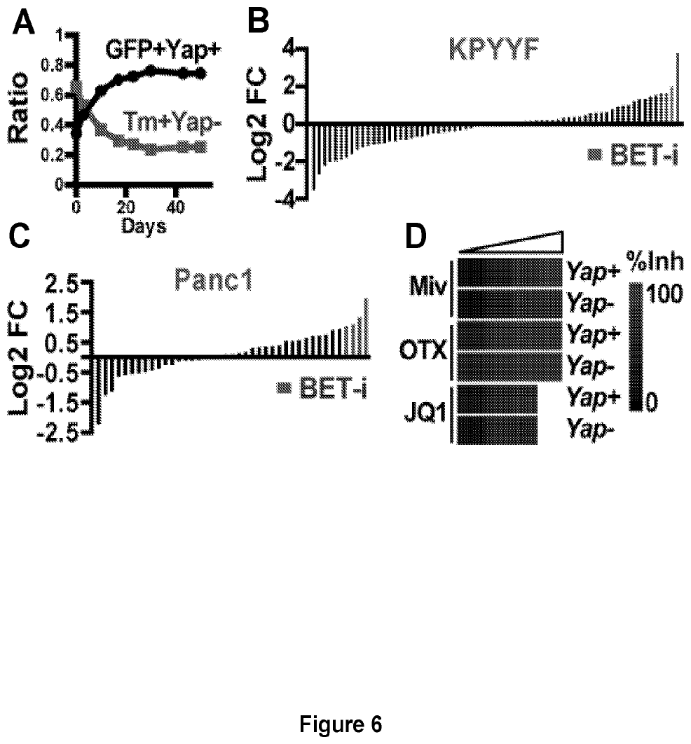

A quantitative FACS-based approach (FIG. 6A) was used to conduct a chemical-genetic screen for epigenetic inhibitors that either promote or prevent the emergence of resistance to YAP loss using cells from KPYYF mice and using Panc1 cells (human pancreas ductal adenocarcinoma cell line). KPYYF mice are KYYF mice that contain an addition p53 mutation. Several BET inhibitors emerged as the top inhibitors that impeded the transition to YAP independence in multiple human and murine PDAC cell lines (FIGS. 6B and 6C). Fully adapted YAP-null PDAC cells showed similar sensitivities to BET inhibitors as parental YAP-WT counterparts (FIG. 6D), suggesting that BET inhibitors block the adaptive reprogramming rather than preferentially kill YAP-null PDAC cells.

### In Primary PDAC Cells, BET Inhibitors Blocked the Expression of Pluripotent Transcription Factors Including SOX2.

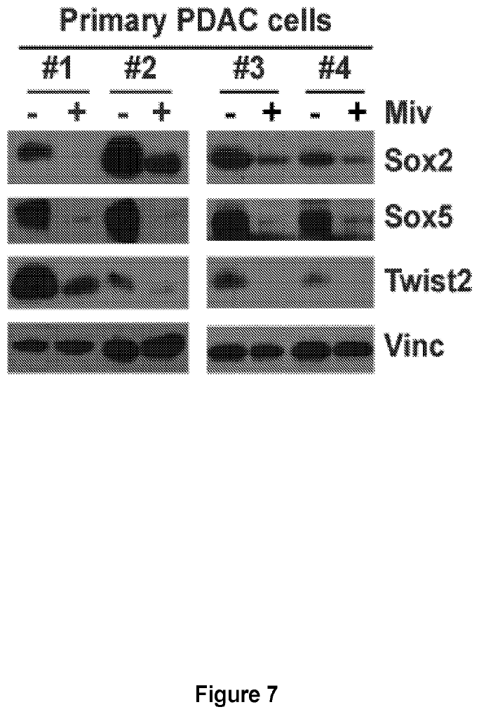

Multiple primary PDAC cell lines were treated with BET inhibitor mivebresib. Using Western blot analysis, it was found that treatment with the BET inhibitor selectively reduced pluripotent transcription factors SOX2, SOX5, and TWIST2 expression after 24 hours as compared to treatment with a control (DMSO) (FIG. 7).

### Inhibition of YAP Sensitized Multiple Cancer Cell Lines to BET Inhibition.

Cell lines of pancreatic cancer (mT4), schwannoma (08031-9), breast cancer (MD-MB-231), kidney cancer (786-O), and liver cancer (HepG2) were depleted of YAP and TAZ mRNAs, which resulted in reduction in YAP/TAZ protein expression. The YAP/TAZ hairpin RNAs were fused with fluorescent protein RFP. These cells were mixed with parental cells that do not express RFP (RFP⁻) and grown together in the presence of BET inhibitor mivebresib or a vehicle control.

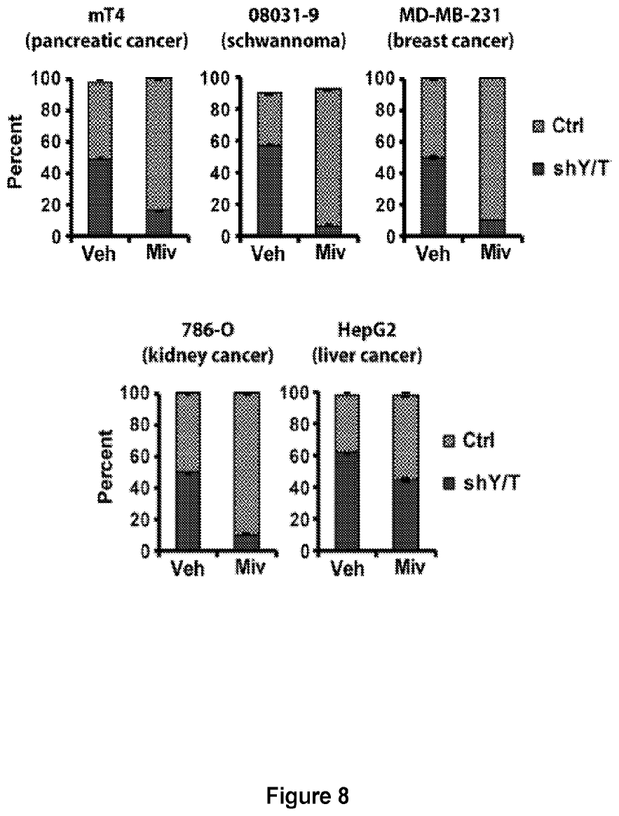

FACS analysis showed that treatment with mivebresib resulted in a greater percentage of control (RFP⁻) cancer cells relative to shY/T (RFP⁺) cancer cells, as compared to treatment with the vehicle. (FIG. 8). These results demonstrate that BET inhibition was sensitized by YAP inhibition, indicating that the effects of YAP inhibition and BET inhibition, and by extension perhaps SOX2 inhibition, are synergistic.

The foregoing description is given for clearness of understanding only, and no unnecessary limitations should be understood therefrom, as modifications within the scope of the invention may be apparent to those having ordinary skill in the art.

Detailed embodiments of the present methods and magnetic devices are disclosed herein; however, it is to be understood that the disclosed embodiments are merely illustrative and that the methods and magnetic devices may be embodied in various forms. In addition, each of the examples given in connection with the various embodiments of the systems and methods are intended to be illustrative, and not restrictive.

Throughout this specification and the claims which follow, unless the context requires otherwise, the word “comprise” and variations such as “comprises” and “comprising” will be understood to imply the inclusion of a stated integer or step or group of integers or steps but not the exclusion of any other integer or step or group of integers or steps.

Throughout the specification, where compositions are described as including components or materials, it is contemplated that the compositions can also consist essentially of, or consist of, any combination of the recited components or materials, unless described otherwise. Likewise, where methods are described as including particular steps, it is contemplated that the methods can also consist essentially of, or consist of, any combination of the recited steps, unless described otherwise. The invention illustratively disclosed herein suitably may be practiced in the absence of any element or step which is not specifically disclosed herein.

The practice of a method disclosed herein, and individual steps thereof, can be performed manually and/or with the aid of or automation provided by electronic equipment. Although processes have been described with reference to particular embodiments, a person of ordinary skill in the art will readily appreciate that other ways of performing the acts associated with the methods may be used. For example, the order of various steps may be changed without departing from the scope or spirit of the method, unless described otherwise. In addition, some of the individual steps can be combined, omitted, or further subdivided into additional steps.

All patents, publications and references cited herein are hereby fully incorporated by reference. In case of conflict between the present disclosure and incorporated patents, publications and references, the present disclosure should control.

### REFERENCES

Collignon J, et al. A comparison of the properties of Sox-3 with Sry and two related genes, Sox-1 and Sox-2. Development 122: 509-520 (1996).

Croci O, et al. Transcriptional integration of mitogenic and mechanical signals by Myc and YAP. Genes Dev. 31: 2017-2022 (2017).

Driskell R, et al. Sox2-positive dermal papilla cells specify hair follicle type in mammalian epidermis. Development. 136: 2815-2823 (2009).

Feng R, et al. Overview of the roles of Sox2 in stem cell and development. Biol Chem. 396: 883-891 (2015).

Fu D, et al. YAP regulates cell proliferation, migration, and steroidogenesis in adult granulosa cell tumors. Endocrine-Related Cancer 21: 297-310 (2014)

Herreros-Villanueva M, et al. SOX2 promotes dedifferentiation and imparts stem cell-like features to pancreatic cancer cells. Oncogenesis 2: e61—e61 (2013).

Ji J, et al. XIAP limits autophagic degradation of Sox2 and is a therapeutic target in nasopharyngeal carcinoma stem cells. Theranostics 8: 1494-1510 (2018).

Liu K, et al. Targeting SOX2 protein with peptide aptamers for therapeutic gains against esophageal squamous cell carcinoma. Mol. Ther.: J. Am. Soc. Gene Ther. 28: 901-913 (2020).

Moroishi T, et al. Glut3 addiction is a druggable vulnerability for a molecularly defined subpopulation of glioblastoma. Cancer Cell 32: 856-868.e5 (2017).

Murakami S, et al. Yes-associated protein mediates immune reprogramming in pancreatic ductal adenocarcinoma. Oncogene 36: 1232-1244 (2017).

Murakami S, et al. A Yap-Myc-Sox2-p53 Regulatory Network Dictates Metabolic Homeostasis and Differentiation in Kras-Driven Pancreatic Ductal Adenocarcinomas. Dev. Cell. 51: 113-128.e9 (2019)

Nowling T K, et al. Identification of the transactivation domain of the transcription factor Sox-2 and an associated co-activator. J. Biol. Chem. 275: 3810-3818 (2000).

Que J, et al. Multiple dose-dependent roles for Sox2 in the patterning and differentiation of anterior foregut endoderm. Development. 134: 2521-2531 (2007).

Ryan D P, et al. Pancreatic adenocarcinoma. N. Engl. J. Med. 371: 1039-1049 (2014).

Schönhuber N, et al. A next-generation dual-recombinase system for time- and host-specific targeting of pancreatic cancer. Nat. Med. 20: 1340-1347 (2014).

Shao D, et al. KRAS and YAP1 converge to regulate EMT and tumor survival. Cell 158: 171-184 (2014).

Singh S, et al. EGFR/Src/Akt signaling modulates Sox2 expression and self-renewal of stem-like side-population cells in non-small cell lung cancer. Mol. Cancer 11: 73 (2012).

Stolzenburg S, et al. Targeted silencing of the oncogenic transcription factor SOX2 in breast cancer. Nucleic Acids Res. 40: 6725-6740 (2012).

Suelves M, et al. DNA methylation dynamics in cellular commitment and differentiation. Brief Funct. Genomics (2016).

Taniguchi J, et al. A synthetic DNA-binding inhibitor of SOX2 guides human induced pluripotent stem cells to differentiate into mesoderm. Nucleic Acids Res. 45: 9219-9228 (2017).

Taranova O V, et al. SOX2 is a dose-dependent regulator of retinal neural progenitor competence. Genes Dev. 20: 1187-1202 (2006).

Sun D, et al. YAP1 enhances cell proliferation, migration, and invasion of gastric cancer in vitro and in vivo. Oncotarget 7: 81062-81076 (2016).

Yin Y, et al. The FBXW2-MSX2-50X2 axis regulates stem cell property and drug resistance of cancer cells. Proc. Natl. Acad. Sci. USA 116: 20528-20538 (2019).

Wang K, et al. FGFR1-ERK1/2-SOX2 axis promotes cell proliferation, epithelial-mesenchymal transition, and metastasis in FGFR1-amplified lung cancer. Oncogene 37: 5340-5354 (2018).

Wuebben E L, et al. The dark side of SOX2: cancer—a comprehensive overview. Oncotarget 8 (2017).

Zanconato F, et al. Genome-wide association between YAP/TAZ/TEAD and AP-1 at enhancers drives oncogenic growth. Nat. Cell Biol. 17: 1218-1227 (2015).

Zanconato F, et al. YAP/TAZ at the Roots of Cancer. Cancer Cell 29: 783-803 (2016).

Zhang X, et al. Pluripotent stem cell protein Sox2 confers sensitivity to LSD1 inhibition in cancer cells. Cell Rep. 5: 445-457 (2013).

Zhao B, et al. Inactivation of YAP oncoprotein by the Hippo pathway is involved in cell contact inhibition and tissue growth control. Genes Dev. 21: 2747-2761 (2007).

Zhao S, et al. SoxB transcription factors specify neuroectodermal lineage choice in ES cells. Mol. Cell Neurosci. 27: 332-342 (2004).

What is claimed is:

1. A method of reducing resistance to the effect of a YAP inhibitor on inducing apoptosis of YAP-dependent cancer cells, the method comprising contacting the YAP-dependent cancer cells with one or more inhibitors of SOX2.

2. A method of reducing resistance to the effect of a YAP inhibitor on inhibiting proliferation of YAP-dependent cancer cells, the method comprising contacting the YAP-dependent cancer cells with one or more inhibitors of SOX2.

3. A method of increasing the efficacy of a YAP inhibitor on inducing apoptosis of YAP-dependent cancer cells, the method comprising contacting the YAP-dependent cancer cells with one or more inhibitors of SOX2.

4. A method of increasing the efficacy of a YAP inhibitor on inhibiting proliferation of YAP-dependent cancer cells, the method comprising contacting the YAP-dependent cancer cells with one or more inhibitors of SOX2.

5. The method of any one of claims 1-4, wherein the YAP-dependent cancer cells are selected from pancreatic ductal adenocarcinoma cells, pancreatic cancer cells, liver cancer cells, sarcoma cancer cells, esophageal cancer cells, glioma cancer cells, schwannoma cells, head and neck cancer cells, non-small cell lung cancer cells, gastric cancer cells, kidney cancer cells, colorectal cancer cells, bladder cancer cells, breast cancer cells, ovarian cancer cells, uterine cancer cells, prostate cancer cells, and melanoma cancer cells.

6. The method of any one of claims 1-5, wherein the YAP-dependent cancer cells are pancreatic ductal adenocarcinoma cells.

7. The method of any one of claims 1-5, wherein the YAP-dependent cancer cells are kidney cancer cells, schwannoma cells, breast cancer cells, or liver cancer cells.

8. The method of any one of claims 1-17, wherein the YAP-dependent cancer cells have a KRAS mutation.

9. The method of any one of claims 1-8, wherein the YAP inhibitor comprises an inhibitor of TAZ, an inhibitor of the YAP/TAZ pathway, an inhibitor of the binding of YAP to TEAD, or an inhibitor of TEAD.

10. The method of any one of claims 1-9, wherein the one or more inhibitors of SOX comprises a bromodomain and extraterminal domain (BET) inhibitor.

11. The method of any one of claims 1-10, wherein the one or more inhibitors of SOX2 contacts the YAP-dependent cancer cells in combination with the YAP inhibitor.

12. The method of any one of claims 1-11, wherein the one or more inhibitors of SOX2 contacts the YAP-dependent cancer cells concurrently with the YAP inhibitor.

13. The method of any one of claims 1-11, wherein the one or more inhibitors of SOX2 contacts the YAP-dependent cancer cells shortly before or shortly after the YAP inhibitor.

14. A method of reducing resistance to the effect of a YAP inhibitor on treating YAP-dependent cancer in a subject, the method comprising administering to the subject one or more inhibitors of SOX2.

15. A method of reducing resistance to the effect of a YAP inhibitor on preventing YAP-dependent cancer in a subject, the method comprising administering to the subject one or more inhibitors of SOX2.

16. A method of increasing the efficacy of a YAP inhibitor on treating YAP-dependent cancer in a subject, the method comprising contacting the YAP-dependent cancer cells with one or more inhibitors of SOX2.

17. A method of increasing the efficacy of a YAP inhibitor on preventing YAP-dependent cancer in a subject, the method comprising contacting the YAP-dependent cancer cells with one or more inhibitors of SOX2.

18. The method of any one of claims 14-17, wherein the YAP-dependent cancer is selected from pancreatic ductal adenocarcinoma, pancreatic cancer, liver cancer, sarcoma, esophageal cancer, glioma, head and neck cancer, non-small cell lung cancer, gastric cancer, kidney cancer, colorectal cancer, bladder cancer, breast cancer, ovarian cancer, uterine cancer, prostate cancer, and melanoma.

19. The method of any one of claims 14-18, wherein the YAP-dependent cancer is pancreatic ductal adenocarcinoma.

20. The method of any one of claims 14-18, wherein the YAP-dependent cancer is kidney cancer, breast cancer, or liver cancer.

21. The method of any one of claims 14-20, wherein the YAP-dependent cancer is associated with a KRAS mutation.

22. The method of any one of claims 14-21, wherein the YAP inhibitor comprises an inhibitor of TAZ, an inhibitor of the YAP/TAZ pathway, an inhibitor of the binding of YAP to TEAD, or an inhibitor of TEAD.

23. The method of any one of claims 14-22, wherein the one or more inhibitors of SOX comprise one or more bromodomain and extraterminal domain (BET) inhibitors.

24. The method of any one of claims 14-23, wherein the one or more inhibitors of SOX2 is administered to the subject in combination with the YAP inhibitor.

25. The method of any one of claims 14-24, wherein the one or more inhibitors of SOX2 is administered to the subject concurrently with the YAP inhibitor.

26. The method of any one of claims 14-24, wherein the one or more inhibitors of SOX2 is administered to the subject shortly before or shortly after the YAP inhibitor.

27. A method of inducing apoptosis of yes-associated protein 1 (YAP)-dependent cancer cells, the method comprising contacting the YAP-dependent cancer cells with one or more inhibitors of YAP and one or more inhibitors of SOX2.

28. A method of inhibiting growth of yes-associated protein 1 (YAP)-dependent cancer cells, the method comprising contacting the YAP-dependent cancer cells with one or more inhibitors of YAP and one or more inhibitors of SOX2.

29. The method of claim 27 or 28, wherein the YAP-dependent cancer cells are selected from pancreatic ductal adenocarcinoma cells, pancreatic cancer cells, liver cancer cells, sarcoma cancer cells, esophageal cancer cells, glioma cancer cells, schwannoma cells, head and neck cancer cells, non-small cell lung cancer cells, gastric cancer cells, kidney cancer cells, colorectal cancer cells, bladder cancer cells, breast cancer cells, ovarian cancer cells, uterine cancer cells, prostate cancer cells, and melanoma cancer cells.

30. The method of any one of claims 27-29, wherein the YAP-dependent cancer cells are pancreatic ductal adenocarcinoma cells.

31. The method of any one of claims 27-29, wherein the YAP-dependent cancer cells are kidney cancer cells, schwannoma cells, breast cancer cells, or liver cancer cells.

32. The method of any one of claims 27-31, wherein the YAP-dependent cancer cells have a KRAS mutation.

33. The method of any one of claims 27-32, wherein the one or more inhibitors of YAP comprises comprise one or more inhibitors of TAZ, one or more inhibitors of the YAP/TAZ pathway, one or more inhibitors of the binding of YAP to TEAD, or one or more inhibitors of TEAD.

34. The method of any one of claims 27-33, wherein the one or more inhibitors of SOX2 comprises a bromodomain and extraterminal domain (BET) inhibitor.

35. The method of any one of claims 27-34, wherein the one or more inhibitors of YAP contact the YAP-dependent cancer cells concurrently with the one or more inhibitors of SOX2.

36. The method of any one of claims 27-35, wherein the one or more inhibitors of YAP and the one or more inhibitors of SOX2 are in the same composition.

37. The method of any one of claims 27-35, wherein the one or more inhibitors of YAP contact the YAP-dependent cancer cells shortly before or shortly after the one or more inhibitors of SOX2.

38. A method of treating YAP-dependent cancer in a subject, the method comprising administering to the subject one or more inhibitors of YAP and one or more inhibitors of SOX2.

39. A method of preventing YAP-dependent cancer in a subject, the method comprising administering to the subject one or more inhibitors of YAP and one or more inhibitors of SOX2.

40. The method of claim 38 or 39, wherein the YAP-dependent cancer is selected from pancreatic ductal adenocarcinoma, pancreatic cancer, liver cancer, sarcoma, esophageal cancer, glioma, head and neck cancer, non-small cell lung cancer, gastric cancer, kidney cancer, colorectal cancer, bladder cancer, breast cancer, ovarian cancer, uterine cancer, prostate cancer, and melanoma.

41. The method of any one of claims 38-40, wherein the YAP-dependent cancer is pancreatic ductal adenocarcinoma.

42. The method of any one of claims 38-40, wherein the YAP-dependent cancer is kidney cancer, breast cancer, or liver cancer.

43. The method of any one of claims 38-42, wherein the YAP-dependent cancer is associated with a KRAS mutation.

44. The method of any one of claims 38-43, wherein the one or more inhibitors of YAP comprises comprise one or more inhibitors of TAZ, one or more inhibitors of the YAP/TAZ pathway, one or more inhibitors of the binding of YAP to TEAD, or one or more inhibitors of TEAD

45. The method of any one of claims 38-44, wherein the one or more inhibitors of SOX2 comprise one or more bromodomain and extraterminal domain (BET) inhibitors.

46. The method of any one of claims 38-45, wherein the one or more inhibitors of YAP are administered to the subject concurrently with the one or more inhibitors of SOX2.

47. The method of any one of claims 38-46, wherein the one or more inhibitors of YAP are administered in the same composition as the one or more inhibitors of SOX2.

48. The method of any one of claims 38-46, wherein the one or more inhibitors of YAP are administered shortly before or shortly after the one or more inhibitors of SOX2.

49. A kit containing a pharmaceutical composition comprising one or more inhibitors of YAP, a pharmaceutical composition comprising one or more inhibitors of SOX2, and a package insert.

50. A kit containing a pharmaceutical composition comprising one or more inhibitors of YAP and one or more inhibitors of SOX2, and a package insert.
